SEQUENCE LISTING (25 sequences)

SEQ ID NO: 1 - DNA, 1749 bp, Infectious salmon anemia virus
ggatccccggcgtttacttcttaaacacgaaagaaatagtgactgcagaagggaaagttgatgaaacaagaggacccttagaaaggacatcagctcctcttatgagagatatctccaggctgatacaagaaacaatagaagaagtggaaacaggaggagacccctctttttcagtaagaagtgaaggaggttctaaaatagaaggaagaatcgccttttcattgcactcagaggtgtccacattgaaaatgaggatagcacttgaacagaaactggccagatatgagtacatgggagaaaaccttctcacacttgtcaaaaacacttctatagacagaatgcagcctgattctgcaatgatggggaaaatggtgttagaaagtcttagaacacacacggtatcctctgagcagttgaatgggagaatgattactgtccaatcgcaaggcctagaaacaatagcgatatcgagtccttttgatgtggaatacgacgatggatacgtattcacaaggatgaaaggagactttgtggcaatcggaagagactacaagggagctatcttatgcttcagagaaggacaagggacattcttcagtgggagaggcaactggtctgggctcatggagagatgtctagttgaaatgagactatgtccatgcttttacagctgcacctggcaagactaccctgacaaaaagagtctctacgaaaaagcaacgttcgaagccaaacagatagtctttgctatgggagaaagtgttggagtagatgtcagagtaaacacagatggtgaaataggagacaagggaatttccttactaacaagggaaagagaggacaaatacatgtcaaaagtgtcttatgagtgcagagtggtcagtgggaaactggtgatgggtttggacaaaatgagcagagtcgcaaaagggaacctagaagtagtgagggaaaaaggagatgacacaagtcagtcagattccttttatgaaggtgtactacaggtaggtagcatgattgggaccacaatggagagtttaaaacagcagttacaaggacctgtgggaatttggagagcatcaggtgtttcagctatggaaaggtgcatgaagagagggcaaagcaaaactgtagtggcaagtgctagatacacattccaaaagatgatggaaaagatggcatctggtagggaagtgtctaaatacagtttgataattgtcatgaggtgctgcattggtttcacgtctgaagctaataagagagctttaactaacatctctggaactggatactacattagtgttgcacaacctaccgtagtaaaactagcaggagaatggttgatcacacctgtgggtaggtctaagacaggggaagttcagtatgtatctgccaagctgaagaaggggatgaccacagggaagctagaattgattaagaaagcagatagatctgacttggacaacttcccagaaccgtcggctgatgaactgttgagagaaggaacaattgtgttaatgcaaatcggaaaagacaaatggttatgtagggtaagaacaggtgataggagagtgaggaccgacacagacatacagagggcagaagcaaaatctcaggttgaaaaagaagatttgatggatgaatatggtgtttaaaataagtggttgtaaaaattgaatgttgtttcttttgctttttgagcctttgacgatacttttaataaataaaatgtccattttgtccgatccc

SEQ ID NO: 2 - protein, 550 aa, Infectious salmon anemia virus
IPGVYFLNTKEIVTAEGKVDETRGPLERTSAPLMRDISRLIQETIEEVETGGDPSFSVRSEGGSKIEGRIAFSLHSEVSTLKMRIALEQKLARYEYMGENLLTLVKNTSIDRMQPDSAMMGKMVLESLRTHTVSSEQLNGRMITVQSQGLETIAISSPFDVEYDDGYVFTRMKGDFVAIGRDYKGAILCFREGQGTFFSGRGNWSGLMERCLVEMRLCPCFYSCTWQDYPDKKSLYEKATFEAKQIVFAMGESVGVDVRVNTDGEIGDKGISLLTREREDKYMSKVSYECRVVSGKLVMGLDKMSRVAKGNLEVVREKGDDTSQSDSFYEGVLQVGSMIGTTMESLKQQLQGPVGIWRASGVSAMERCMKRGQSKTVVASARYTFQKMMEKMASGREVSKYSLIIVMRCCIGFTSEANKRALTNISGTGYYISVAQPTVVKLAGEWLITPVGRSKTGEVQYVSAKLKKGMTTGKLELIKKADRSDLDNFPEPSADELLREGTIVLMQIGKDKWLCRVRTGDRRVRTDTDIQRAEAKSQVEKEDLMDEYGV

SEQ ID NO: 3 - DNA, 2185 bp, Infectious salmon anemia virus
ccatggccgcgggattgaacgctctttaataaccatggaaactctagtaggagggctgctgactggagaagattctctgatcagtatgtcaaacgatgtatcttgtctttatgtttacgatggaccaatgagagttttctctcagaacgcattaatgccaactctgcaaagtgtaaaaagaagtgaccaattttccaaagggaaaacaaagagatttatcattgacctgttcggaatgaagagaatgtgggacatcggaaacaaacagttggaagacgagaacttagacgaaactgtaggcgtggctgacttggggctggtgaaatatctaatcaacaacaagtacgatgaagcagaaaagacaagtttaaggaagtcaatggaagaagcattcgaaaaatccatgaacgaagaatttgtggttttaaacaaaggaaagtctgcaaacgacatcatttcagacacaaatgcgatgtgcaaattctgtgtaaagaactggatagtggcaacaggtttcaggggaagaacgatgtcagatttaattgaacaccatttcagatgcatgcaagggaaacaggaggtgaaaggatacatttggaaacacaagtacaacgaaaggcttaaaagaaaacagctaagcaaagaagaagtgaaattcgacagagaagaatatacttcaagaagcttcagactactctctttcttgaagaacagcgagaggaccaaactcgagccgagagcagtgttcacagcaggagttccatggagggcattcatcttcgtcctagaacagacaatgctggtggtaaacaaactggacccgaattcagtgatatggatgggaagtgatgcaaagataaacaccacaaactccaggataaaggaaatagggatgaaaaatcaaggacaaacactagtgacactcacaggagataactccaaatacaacgagagcatgtgcccagaggtgatgatggtgttcctaagagaactaggaataaaaggaccaatgttggaagtactggactatgcgctgtggcaattttcacagaagagtgtaaaacctgtcgcacctataaagaagagaaccggcaagtctaccgtggtgataaaagcagattccgttaaggagtgtagagatgccttcaacgaaaaggaactggagctgattcaaggagttgaatggatggacgacggatttgtgagagtgaggagaggaatgttgatgggaatggcaaacaacgcttttaccacagcttctacaattgcctcctcttttagtttcacaccagaagctgtgtacacattacagagctcagacgacttcgttacaggtagctgtggaagagacgtgcaacacgcaagacaaaggctagagatggctcttaaagtgagcaaagccgcaggtctgaacgtatcacagaagaagtcattctacgttgaagggacaactttcgagttcaactctatgttcgtaagagacggtaaagtgatggcaaacggaggaaactttgagaacatgacagttcctggaggattaggaccatctacagatctctttgtcgtggggaaacaagcaagaaactccatgttgagaggcaacctatccttcagccaggcgatggagatgtgcaaaataggaatcacaaatgttgagaaagtttactatggaaacagaaaataccaggagctgaaaaatgagataagagagaaatgtggagaagaaacgatgtccataccagagagcatgggaggagacaggaaaccaagaccgtgggaattacctcagagctttgatggaattgccttaaaagaagctgtgaacagaggacattggaaagctgccaagtacatcaaatcttgctgcagcatagagttcgatgaagaaggagaccaatcttgggacacttcgaaaacagcacttgtggtcataaggaaaaatgaaacggacatgagaagaagaactgttaaaacgaggaacccaaaagataaaatcttcaatgatgcaatgaacaaggccaaaaggatgtacgaaacagtcgtggacagaaacccattactaggtctgaaggggaagggaggtagactgacagtaaaagacttgaaagcaaggaagcttattgatgaagtagaagttgttaagaagaaaaagcatgtttgaaatcactagtgcggccgcctgcag

SEQ ID NO: 4 - protein, 726 aa, Infectious salmon anemia virus
HGRGIERSLITMETLVGGLLTGEDSLISMSNDVSCLYVYDGPMRVFSQNALMPTLQSVKRSDQFSKGKTKRFIIDLFGMKRMWDIGNKQLEDENLDETVGVADLGLVKYLINNKYDEAEKTSLRKSMEEAFEKSMNEEFVVLNKGKSANDIISDTNAMCKFCVKNWIVATGFRGRTMSDLIEHHFRCMQGKQEVKGYIWKHKYNERLKRKQLSKEEVKFDREEYTSRSFRLLSFLKNSERTKLEPRAVFTAGVPWRAFIFVLEQTMLVVNKLDPNSVIWMGSDAKINTTNSRIKEIGMKNQGQTLVTLTGDNSKYNESMCPEVMMVFLRELGIKGPMLEVLDYALWQFSQKSVKPVAPIKKRTGKSTVVIKADSVKECRDAFNEKELELIQGVEWMDDGFVRVRRGMLMGMANNAFTTASTIASSFSFTPEAVYTLQSSDDFVTGSCGRDVQHARQRLEMALKVSKAAGLNVSQKKSFYVEGTTFEFNSMFVRDGKVMANGGNFENMTVPGGLGPSTDLFVVGKQARNSMLRGNLSFSQAMEMCKIGITNVEKVYYGNRKYQELKNEIREKCGEETMSIPESMGGDRKPRPWELPQSFDGIALKEAVNRGHWKAAKYIKSCCSIEFDEEGDQSWDTSKTALVVIRKNETDMRRRTVKTRNPKDKIFNDAMNKAKRMYETVVDRNPLLGLKGKGGRLTVKDLKARKLIDEVEVVKKKKHVNHCGRLQ

SEQ ID NO: 5 - DNA, 2046 bp, Infectious salmon anemia virus
agcaaagattgctcaaatcccaaaaataatacagaaaacgtataagagatggccgataaaggtatgacttattcttttgatgtcagagacaacaccttggttgtaagaagatctaccgctactaaaagtggcattaagatctcctacagagaggatcgaggaacatcacttctccaaaaggcattcgccgggacagaagatgaattctgggtggagttagatcaagatgtctacgttgacaaaaagattagagaattcctggtagaagagaaaatgaaggacatgagcacaagagtgtctggggcagtggcagcagcaattgaaagatcagttgaatttgacaatttctcaaaagaagcagcagctaacattgaaatggctggtgtagatgatgaagaagctggaggaagtggtctggtagacaacagaaggaagaacaaaggggtctcaaacatggcctacaatctgtctctattcatagggatggtgtttcctgctctcactactttcttcagtgctatcctatcagaaggtgaaatgagcatctggcaaaatggacaagcaatcatgagaattctggcactggcagatgaagacggaaagagacaaacaagaacaggaggacagagggtggacatggctgatgtaaccaagctgaacgtagtcacggctaacggaaaagtcaagcaagttgaagtaaacttgaacgatctcaaagcagcattcaggcagagtagacctaaaagatcggactacagaaaagggcaaggttccaaggctacagaatcaagcatctccaaccaatgtatggcactgattatgaaatctgtgctgtcagcagaccaactttttgctccgggagtgaagatgatgaggacgaacggtttcaatgcgtcgtacacaacactggcagaaggggcaaacattccgagcaagtacctaagacacatgaggaactgcggaggagtagctctggacctgatgggaatgaagaggatcaaaaactcacctgaaggagccaagtctaagatcttttccatcatccagaagaaagtaagaggaagatgtcgcacagaggagcaacgcctcctgactagcgcactgaaaatcagcgacggtgaaaacaagttccagagaatcatggacactctatgtacaagcttcctgattgaccctccaagaactaccaaatgcttcattccacctatttccagtctcatgatgtacatccaagaaggcaactctgtactggcaatggatttcatgaaaaacggagaggacgcctgcaagatctgcagagaagccaaactgaaagtgggggtaaacagtacgttcacaatgtcagtagctagaacatgcgttgcagtgtcaatggttgcaacagctttttgttctgcagatatcatcgagaatgcagtgcctggttccgaaaggtacagatccaacatcaaggctaacacaaccaaaccaaaaaaggactccacttacacaattcaaggacttagattgtctaacgtgaggtatgaagcaagacctgaaacatcacaaagcaacacagacagaagttggcaagtgaacgtgactgacagcttcggaggacttgctgtgttcaaccaaggggcaattagagaaatgctaggagacggaacatcagagacaactagtgtgaacgtcagagccctggtgaagagaattctgaaatcagcttcagagaggagtgcaagagctgtaaagacatttatggtgggagaacaagggaaatcagctattgttatctctggtgtgggactgttctctattgactttgaaggggtagaggaagcggaaagaataactgacatgacacctgaaattgagtttgacgaggacgacgaggaagaggaagacattgacatttagagtgacaattatgtaacttcctaattaccctatattgtttgaatatataatgaaactattgtgtgttaaaggttgtgggtttgattattaaatttaaattgaaacggtattgacgatatttacaaaaaaaaaaaaaaaaaaaaaaaa

SEQ ID NO: 6 - protein, 616 aa, Infectious salmon anemia virus
MADKGMTYSFDVRDNTLVVRRSTATKSGIKISYREDRGTSLLQKAFAGTEDEFWVELDQDVYVDKKIREFLVEEKMKDMSTRVSGAVAAAIERSVEFDNFSKEAAANIEMAGVDDEEAGGSGLVDNRRKNKGVSNMAYNLSLFIGMVFPALTTFFSAILSEGEMSIWQNGQAIMRILALADEDGKRQTRTGGQRVDMADVTKLNVVTANGKVKQVEVNLNDLKAAFRQSRPKRSDYRKGQGSKATESSISNQCMALIMKSVLSADQLFAPGVKMMRTNGFNASYTTLAEGANIPSKYLRHMRNCGGVALDLMGMKRIKNSPEGAKSKIFSIIQKKVRGRCRTEEQRLLTSALKISDGENKFQRIMDTLCTSFLIDPPRTTKCFIPPISSLMMYIQEGNSVLAMDFMKNGEDACKICREAKLKVGVNSTFTMSVARTCVAVSMVATAFCSADIIENAVPGSERYRSNIKANTTKPKKDSTYTIQGLRLSNVRYEARPETSQSNTDRSWQVNVTDSFGGLAVFNQGAIREMLGDGTSETTSVNVRALVKRILKSASERSARAVKTFMVGEQGKSAIVISGVGLFSIDFEGVEEAERITDMTPEIEFDEDDEEEEDIDI

SEQ ID NO: 7 - DNA, 1787 bp, Infectious salmon anemia virus
caagatggataacctccgtgaatgcataaaccgcaaaagaagactacttgccttaccagatgttcctgaaacttcggatgcctttctaagtgatttgagacatctatacatgtgtgttgctttctgtgatcaacacaaaaccactggagacgaatcaagattcaccaacctggaattacttgaccaagatgaagcactaggtgcccaaagagcttttgaagccaaacatggaataaaaggaggttctttaggagacgttcttgaccatgaactgaaaaaggtcattgaatttacttttacttctggaagtttgtatattgccgaacaaagaaaaagaaagactcaagcagactcaataattgtgtgcgtttcagaaggacttaacgacttcagcgtatcacacggagtgctagacatgggacttgtggaaacaggggtgaatgcagtaagagatttctgcacacaaaacggaataccaatgaagataaatcaggtaggatccacgagaacaccaacaccgatcagcacatgcaaaatctctgaacaaataacacgacarataaacagtacaattactgaaaggaaaatggaaacagtactggcagcaatcgcaattaaaccagaactcaaayyaactcagaaaggatgcagmmcttgtaaagaactagaagatgaaaatattctgtggatggaccctcaattctgtgaaattgatgaaagttttccttacagaggagggccatacgggaacttcctgcaagaattgctgcttacaaccaacgacgtagagaccaacgggaaagacagagaagaagtagtaaagaasatactggataacaaggcgttcaccgttgaaagtggtgaatgcataataacacttccagacaaaatgacttgtttcggagaacargagaagaagagaccagcaacaatagacgaagtgagaaccgcaggagaaaggtttgaacagagtgttaaaccgaaaacccaaagatatggaaggttatcagacaaatggatggagcttgaaaagtttatctttactgcaagcaaaacagaagtggatactttcctttctgtagggaccgaaagacttgagtcggttggagtgtgtgtcggagctttacacagagcgaccacaaccaggataattagacctatgattcaaggagggaaatgttgggggatgatgttcaaaacaaagtccaaaatgggagacacgaggaaggaaggatactgtcacgcaatcattttcggaaaaggggaagataaatcaggacaaaacaagatgacaatgatggggaaaacagtacattggcatctaagagtagttaagtctaaaggagactggatggcgcaacaactctgtgcaaacaaaagcagaatatggcaacatgaccctgagctagtaacagaaggagtgacagttctaatgacgcctttttctcagaaaattgcaaccattagtagatggagggcaatgaggttagacagcatgtttcatgtttctagtgcctggcatcattcacctgcgtgtgaagctgcatcggcaatgctgagaaagtttgtggagatagtacatgccatcaaccagaaaagagattggggtgttgtggggagtatggaggacatggtgaaggaagtggaggaaataggggagcacttgcagacggcatgtgaytttagagtttacaacatktgcaaagccttgattcagaaaattgcagtcagtacccaatgagtggttatttacttgtaaattgttgtgtgtttgacgatatgtattt

SEQ ID NO: 8 - protein, 578 aa, Infectious salmon anemia virus
  misc_feature (210)..(210): Xaa can be any naturally occurring amino acid
MDNLRECINRKRRLLALPDVPETSDAFLSDLRHLYMCVAFCDQHKTTGDESRFTNLELLDQDEALGAQRAFEAKHGIKGGSLGDVLDHELKKVIEFTFTSGSLYIAEQRKRKTQADSIIVCVSEGLNDFSVSHGVLDMGLVETGVNAVRDFCTQNGIPMKINQVGSTRTPTPISTCKISEQITRQINSTITERKMETVLAAIAIKPELKXTQKGCXXCKELEDENILWMDPQFCEIDESFPYRGGPYGNFLQELLLTTNDVETNGKDREEVVKXILDNKAFTVESGECIITLPDKMTCFGEQEKKRPATIDEVRTAGERFEQSVKPKTQRYGRLSDKWMELEKFIFTASKTEVDTFLSVGTERLESVGVCVGALHRATTTRIIRPMIQGGKCWGMMFKTKSKMGDTRKEGYCHAIIFGKGEDKSGQNKMTMMGKTVHWHLRVVKSKGDWMAQQLCANKSRIWQHDPELVTEGVTVLMTPFSQKIATISRWRAMRLDSMFHVSSAWHHSPACEAASAMLRKFVEIVHAINQKRDWGVVGSMEDMVKEVEEIGEHLQTACDFRVYNXCKALIQKIAVSTQ

SEQ ID NO: 9 - DNA, 1504 bp, Infectious salmon anemia virus
agttaaagatggcttttctaacaattttagtcttgttcctttttaaagaggttctttgtgaaccttgtatttgtgagaacccaacatgtctaggaataacaatcccacaggcaggtttcgtaagaagcgctccaggaggtgtacttctaactgagacaatcacggaaagaccacaactaacagagtggacaacctccagaccgaagcttgaagaaactctctggttagatggggaaacaaagaacggaaaagtatctcagacactattcgaagccatccaaggtacacagatggagaactgtgcagtgaaagctgtgttagacacaacatttgtcaacctaaccaaacaagacattgtgctaggaaaaatcaaggtgtctgagtttggtggagacagtgacatttccaaatgtggaagaaaaggactaaaggttttcatctgtggaggtactgttggatacgtgacaagaggatgcccacctgaggagtgcaaaggaaagaaagggagaatgatggctctcgaacccactacggattgtggtgtcgaaaaaggacttacaactgacagaatcaaaacaggaatgttggacatcacaagttgctgtacacaacatggatgcacaaagggaatcagagtagaggttccttcaccagtacttgtatcttcaaaatgtcaagaagtcactttcagagtggttccattccattcagtacctgacaagctagggtttgcacgcacaagctcattcacactaaaagctaacttcgtgaacaaacatgggtggtccaagtataatttcaacctaagaggatttcctggagaagagttcattaagtgttgtggatttacgttgggagtcggaggagcgtggtttcaagcctacttaaatggaatggttcaaggtgacggtgccgcatctgcagacgacgtgaaagagaaactcaacggaataatcgaccagataaacaaagcgaacacacttcttgaaggagaaattgaagcagtgaggaggattgcctatatgaaccaagcatcaagtcttcagaaccaagtggaaatcggactaataggtgaatatttgaacattagcagttggttggagactactacattaactaaaacagaagaaggcttgatgaagaatggctggtgtcagtctaacacgcactgctggtgtccacctaaacctacaattgttcccaccattggatatgttgacagtataaaagaagtaacgggtacaagttggtggatggttatgatacattacattattgtggggttaatagttattgtggtggtggtgtttggtttaaaactatggggatgtcttagaaggtgaaatgtcggtctaaaaattctttttctgtacattactaaagggtagcttaaccaaggtgtttatgtatatagactattattggataagttagaaatttgtatctgattatgcattattaattgtataaatagaatcactagtgcggccgcctgcaggtcgac

SEQ ID NO: 10 - protein, 497 aa, Infectious salmon anemia virus
LKMAFLTILVLFLFKEVLCEPCICENPTCLGITIPQAGFVRSAPGGVLLTETITERPQLTEWTTSRPKLEETLWLDGETKNGKVSQTLFEAIQGTQMENCAVKAVLDTTFVNLTKQDIVLGKIKVSEFGGDSDISKCGRKGLKVFICGGTVGYVTRGCPPEECKGKKGRMMALEPTTDCGVEKGLTTDRIKTGMLDITSCCTQHGCTKGIRVEVPSPVLVSSKCQEVTFRVVPFHSVPDKLGFARTSSFTLKANFVNKHGWSKYNFNLRGFPGEEFIKCCGFTLGVGGAWFQAYLNGMVQGDGAASADDVKEKLNGIIDQINKANTLLEGEIEAVRRIAYMNQASSLQNQVEIGLIGEYLNISSWLETTTLTKTEEGLMKNGWCQSNTHCWCPPKPTIVPTIGYVDSIKEVTGTSWWMVMIHYIIVGLIVIVVVVFGLKLWGCLRRNVGLKILFLYITKGLNQGVYVYRLLLDKLEICILCIINCINRITSAAACRS

SEQ ID NO: 11 - DNA, 1323 bp, Infectious salmon anemia virus
agcaaagatggcacgattcataattttattcctactgttggcgcctgtttacagtcgtctatgtcttagaaaccatcctgacaccacctggataggtgactcccgaagcgatcaatcaagggtgaaccaacagtctcttgatctggttacaaacttcaagggaattctacaagccaagaacgggaatggtctcatgaagcagatgagcggaaggttcccaagtgattggtaccaacctactacaaagtataggattctatacattggtacaaacgactgcactgagggccctaacgacgtgatcataccgacgtcaatgacactagacaatgtggcaagggacctgtacctgggagcatgtcgaggagatgtaagagtgacaccaaccttcgtgggagcagctgagcttggactgattgggagaacagatgccttaacagaattttctgtaaaggtgctgactttcaacaaccctactattgtagtagttggactaaatggaatgtcaggaatctacaaggtctgcattgctgcctcttctggaaacgtaggcggagtcaacttggtgaacggatgcggatacttcagcgctcctctgagattcgacaacttcaaaggacagatctacgtgtcagacacctttgaagtcagaggaacaaagaacaaatgtgtcatacttagatcttctagcaatgctcctttgtgtacacatatcaaaagaaacattgagttggatgagtacgttgacacaccaaacactgggggcgtatatccttctgatgggtttgattctcttcacggctctgcttcgattagaacttttttaacagaggcactgacatgtccaggtgtagattgggacagaattgatgcagcttcatgcgagtatgacagttgtcctaaacttgtgaaagaatttgaccaaacagggctcggaaacacagatactcaaataatgagagagctagaagcacaaaaggagatgattggtaaacttggcagaaacattacagacgtaaacaacagagtagatgctattccaccacagcttagcaacatcttcatctctatgggagtggcaggttttgggatagcactgtttctagcagggtggaaggcttgtgtttggatagcagctttcatgtataagtctagaggtagaaacccacctgcaaatctgtctgttgcttgatactaagacaaacaaagttttcaaataatcaaatgttttctaatgtaatgtaaaattcaaatcgtatgtgatattattattttgaagacgttcttgatgttgtacgtttacagaaaagtgcattttttact

SEQ ID NO: 12 - protein, 394 aa, Infectious salmon anemia virus
MARFIILFLLLAPVYSRLCLRNHPDTTWIGDSRSDQSRVNQQSLDLVTNFKGILQAKNGNGLMKQMSGRFPSDWYQPTTKYRILYIGTNDCTEGPNDVIIPTSMTLDNVARDLYLGACRGDVRVTPTFVGAAELGLIGRTDALTEFSVKVLTFNNPTIVVVGLNGMSGIYKVCIAASSGNVGGVNLVNGCGYFSAPLRFDNFKGQIYVSDTFEVRGTKNKCVILRSSSNAPLCTHIKRNIELDEYVDTPNTGGVYPSDGFDSLHGSASIRTFLTEALTCPGVDWDRIDAASCEYDSCPKLVKEFDQTGLGNTDTQIMRELEAQKEMIGKLGRNITDVNNRVDAIPPQLSNIFISMGVAGFGIALFLAGWKACVWIAAFMYKSRGRNPPANLSVA

SEQ ID NO: 13 - DNA, 966 bp, Infectious salmon anemia virus
tacaaagaaaatgttcagaacatgtctggatttaacttcgaggtaatggtgccggaacaaggaggaaaagtggtcttcagccttactgaaacggggtcatgtgtctcgttttacggagatgatgaaccaggtgaagggtcctgcgaacttgcctctgaaaacatggattttccaagttgtcctctggggaatggagatgacttctgtctgtcgctggcgctaagcacaatgagatggtctgggatgaccaagagaaacaacttcatggacagattcattggaagttttgttcattgtacaccagtgatgatctggtcgtatggaaatttgtccaagaaaagccatcacaaaatggtttgccacacttgcccagacgagtacaagttcagtgacaaggacgagatgcagggatactatgaggaatgtctagaggcttctactgacattttccttgatgaacttgctactgttgttacaggtggcttctttcctgtcggactcaaaggttcctggggaggatggtacctcaagtacgtcaggtatgctggacctcttgcgggatcaagtggattcattgtcaatcaacgattctacgacagagcccaaaacaagactggatccagggttgtatccatggttgaaatggacggagacggcttatcgttcatctacgagaagcctagcgtctaccatagtgatgggtgcactggttcagcagcgaggttctggaaacgggatcacaatgagagagctggagttgagcttagggctggacttcacttcagaatgtgattggttgaaaacttgttatgtaaacaagaattttgtgtttttgtcagaaaaagaaattgctgtaaacatggaagttgaaaaattcatttgtaatgagaactaaagatgtctttgtgttcaaattttaactaatgacaatatatgaaatatgtcgtacatggtgttgatgataat

SEQ ID NO: 14 - protein, 256 aa, Infectious salmon anemia virus
MSGFNFEVMVPEQGGKVVFSLTETGSCVSFYGDDEPGEGSCELASENMDFPSCPLGNGDDFCLSLALSTMRWSGMTKRNNFMDRFIGSFVHCTPVMIWSYGNLSKKSHHKMVCHTCPDEYKFSDKDEMQGYYEECLEASTDIFLDELATVVTGGFFPVGLKGSWGGWYLKYVRYAGPLAGSSGFIVNQRFYDRAQNKTGSRVVSMVEMDGDGLSFIYEKPSVYHSDGCTGSAARFWKRDHNERAGVELRAGLHFRM

SEQ ID NO: 15 - protein, 146 aa, Infectious salmon anemia virus
MNLLLLLQVASFLSDSKVPGEDGTSSTSGMLDLLRDQVDSLSINDSTTEPKTRLDPGLYPWLKWTETAYRSSTRSLASTIVMGALVQQRGSGNGITMRELELSLGLDFTSECDWLKTCYVNKNFVFLSEKEIAVNMEVEKFICNEN

SEQ ID NO: 16 - DNA, 736 bp, Infectious salmon anemia virus
tgcaaagattggctatctaccatgcatgagagaagcaaacccaaaaccacgggagctgatcagacatgccttgaagaagaaaaagagaccagaggtggtttacgcaatgggagttcttctgacactggggggagagagcggactgaccgtggagtttcctgttccagaaggaaaaactgtgaaggtcaaaaccttgaaccaattggtgaacgggatgatcagtcgagcgacgatgaccctctactgtgtgatgaaagatccaccatcgggaggcatggcaacgctgatgagagaccacatcaggaactggctgaaggaggaatcaggatgccaggacgcggatggtggagaggaaaaatgggcaatggtgtatggtatgatttcacccgacatggcagaggagaagacgatgctgaaggagctgaaaacaatgctacacagcaggatgcagatgtatgctctgggtgcaagttcgaaagccctagagaatttagaaaaggccatcgtcgctgcagttcatcgacttccggcatcctgctcgacagagaagatggtgcttctggggtacctgaagtaagcttcaaagaaagaatggaagcggagaagaagaaactgaaagagctggacgacaagatctacaagctaaggagaagattgaggaagatggagtacaagaaaatggggatcaaccgagaaatcgacaaattggaagactctgtacaataaaatcactagt

SEQ ID NO: 17 - protein, 234 aa, Infectious salmon anemia virus
MHERSKPKTTGADQTCLEEEKETRGGLRNGSSSDTGGRERTDRGVSCSRRKNCEGQNLEPIGERDDQSSDDDPLLCDERSTIGRHGNADERPHQELAEGGIRMPGRGWWRGKMGNGVWYDFTRHGRGEDDAEGAENNATQQDADVCSGCKFESPREFRKGHRRCSSSTSGILLDREDGASGVPEVSFKERMEAEKKKLKELDDKIYKLRRRLRKMEYKKMGINREIDKLEDSVQ

SEQ ID NO: 18 - protein, 183 aa, Infectious salmon anemia virus
MREANPKPRELIRHALKKKKRPEVVYAMGVLLTLGGESGLTVEFPVPEGKTVKVKTLNQLVNGMISRATMTLYCVMKDPPSGGMATLMRDHIRNWLKEESGCQDADGGEEKWAMVYGMISPDMAEEKTMLKELKTMLHSRMQMYALGASSKALENLEKAIVAAVHRLPASCSTEKMVLLGYLK

SEQ ID NO: 19 - DNA, 31 bp, Artificial Sequence
  Oligonucleotide Primer
aagcagtggtaacaacgcagagtagcaaaga

SEQ ID NO: 20 - DNA, 21 bp, Artificial Sequence
  Oligonucleotide Primer
gaacgctctttaataaccatg

SEQ ID NO: 21 - DNA, 19 bp, Artificial Sequence
  Oligonucleotide Primer
tcaaacatgctttttcttc

SEQ ID NO: 22 - DNA, 19 bp, Artificial Sequence
  Oligonucleotide Primer
agcaaagatggcacgattc

SEQ ID NO: 23 - DNA, 24 bp, Artificial Sequence
  Oligonucleotide Primer
tgcacttttctgtaaacgtacaac

SEQ ID NO: 24 - DNA, 35 bp, Artificial Sequence
  Oligonucleotide Primer
aagcagtggtaacaacgcagagtctatctaccatg

SEQ ID NO: 25 - DNA, 19 bp, Artificial Sequence
  Oligonucleotide Primer
ttattgtacagagtcttcc

(19) United States
(12) Patent Application Publication
(10) Pub. No.: US 2004/0147467 A1
(43) Pub. Date: Jul. 29, 2004

(54) Nucleic acids encoding isav polypeptides

(76) Inventors: Sharon C. Clouthier, Orono, ME (US); Eric D. Anderson, Orono, ME (US)

Correspondence Address: KLARQUIST SPARKMAN, LLP, 121 SW SALMON STREET, SUITE 1600, PORTLAND, OR, 97204, US

(21) Appl. No.: 10/472,990

(22) PCT Filed: Mar. 29, 2002
(86) PCT No.: PCT/US02/09681

(51) Int. Cl. A61K 48/00; C07H 21/04

(52) U.S. Cl. 514/44; 536/23.5

(57) ABSTRACT

Infectious Salmon Anemia Virus (ISAV) nucleic acid molecules and polypeptides are disclosed, as well as host cells and transgenic fish transformed by expression vectors containing such nucleic acids. The nucleic acid molecules can encode antigenic epitopes capable of eliciting an immune response in a host cell or animal, such as an immune response against ISAV, and the polypeptides themselves can be antigenic epitopes and also induce such an immune response.

### FIELD

This invention relates to Infectious Salmon Anemia Virus (ISAV), more specifically to ISAV nucleic acid sequences and the peptides these nucleic acids encode. This invention also relates to the use of ISAV peptides in producing an immune response in fish.

### BACKGROUND

Global aquaculture production is estimated at 39.4 million tons annually, is worth $52.5 billion (US), and contributes over 20% of the total fish harvest. Although the United States contributes only 2% of global production, the aquaculture industry in this country is gaining momentum and importance. For example, farm-raised salmon are a prominent industry in the Pacific Northwest and Maine.

As the natural fisheries provided by the open seas decline globally, and the world's population is projected to grow to 8 billion people by 2025, cultured finfish products will be in increasing demand as an important protein source. Some of the factors that must be successfully accommodated to sustain the economic viability and increase the productivity of finfish culture include maintaining adequate culture facilities, complying with regulatory and environmental requirements and countering the many infectious pathogens and diseases that can threaten farmed populations of aquatic animals. Of these variables, the economic impact of disease on cultured finfish operations has become increasingly important. One of the primary means for raising finfish culture efficiency is through the development of reliable treatments against infectious pathogens and thus improve the overall health of farmed species.

Infectious salmon anemia (ISA), formerly called Hemorrhagic Kidney Syndrome (HKS), has caused massive economic losses in the Atlantic salmon farming industry in Norway, Atlantic Canada, and Scotland. Mortality from ISA disease is variable, ranging from 10% to more than 50%. Clinical signs of the disease are apparent in Atlantic salmon, but other salmonids can act as non-symptomatic reservoirs for the virus. The pathological changes associated with ISA are characterized by severe anemia, leukopenia, ascites and hemorrhaging of internal organs with subsequent necrosis of hepatocytes and renal interstitial cells. The infectious agent is an enveloped virus (ISAV) which replicates in endothelial cells in vivo and buds from the cell surface. The virus has a single-stranded RNA genome consisting of 8 segments with negative polarity, and the structural, morphological, and physiochemical properties of the virus suggest that ISAV is related to members of the Orthomyxoviridae family (see, e.g., Falk, et al., J. Virol. 71:9016-23 (1997)).

ISA originally appeared in Norway in 1984 (Thorud and Djubvik, 1988). In 1996 and 1998, the disease was diagnosed on fish farms in Atlantic Canada and Scotland, respectively. Subsequent to the appearance of clinical disease in Canada, ISAV surveillance programs were instituted in New Brunswick. A central aspect of the Canadian ISAV management approach involves the depopulation of ISAV-infected cages that are found through participation in the surveillance protocols. The Canadian government and Canadian salmon producers themselves have developed several compensation programs to offset losses from eradication measures, which has helped lower the incidence of new cases of both virus and disease at previously negative marine sites. Recent Canadian outbreaks are currently confined to the Bay of Fundy area of Maritime Canada. However, the Norwegian disease pattern has shown that the virus spreads from population to population principally by exposure to body fluids from infected fish, through untreated water coming from fish processing plants or through shared equipment that hasn't been properly disinfected at marine sites. Thus, Atlantic salmon netpens at neighboring Maine marine sites are at considerable risk of encountering ISA virus.

Historically, the elimination of ISA disease in other countries through the attempted eradication of ISA virus has proven to be futile. Given the many unknown factors involved in disease transmission, including ties between the ISA pathogen and wild reservoirs of virus, outright elimination of ISA and the virus (ISAV) does not appear to be an achievable goal. However, as shown over time in several other international epizootics of ISA, mortality from ISA can be decreased through the development of biosecurity protocols and good management techniques. Nonetheless, the development of effective treatments against ISAV remains a high priority for salmon producers in the U.S. and elsewhere.

Fish that survive ISA demonstrative a protective immune response indicating that prophylactic treatment against ISA is possible. Whole killed viral formulations have been shown to be effective against other viral diseases of fish, but the disadvantage of such an approach is that virulent virus may remain in the formulation if extreme care is not taken during the manufacturing process. Additionally, the immune response conferred is often brief and may need to be boosted. Finally, killed virus formulations are prepared by growing virus in large amounts in cell culture or in the actual animal species, and either method is expensive. Furthermore, if the titer of the amplified virus is low, then achieving the appropriate antigenic dose within the final formulation requires the addition of more virus and raises the cost of production. Thus, a need remains for an effective ISAV vaccine.

### SUMMARY

ISAV nucleic acid molecules are disclosed. In some embodiments, the nucleic acid molecule has a sequence at least 70% identical to SEQ ID NO: 1, a nucleic acid sequence at least 85% identical to SEQ ID NO: 3, or a nucleic acid sequence at least 85% identical to SEQ ID NO: 11, or a sequence consisting essentially of SEQ ID NO: 1, SEQ ID NO: 3, or SEQ ID NO: 11. In particular embodiments, the nucleic acid molecule is operably linked to a heterologous nucleic acid, such as an expression control sequence. In one specific non-limiting example, the nucleic acid sequence is included in a vector.

Host cells and transgenic fish transformed by such nucleic acids also are disclosed. In some embodiments, the nucleic acid molecule encodes an antigenic epitope capable of eliciting an immune response in the cell or fish, such as an immune response against ISAV. Particular fish and fish cells include (but are not limited to) rainbow trout, coho salmon, chinook salmon, amago salmon, chum salmon, sockeye salmon, Atlantic salmon, arctic char, brown trout, cutthroat trout, brook trout, catfish, tilapia, sea bream, seabass, flounder, or sturgeon.

### BRIEF DESCRIPTION OF THE DRAWINGS

FIG. 1 is a graph showing the results of an efficacy trial of Atlantic salmon treated with whole killed ISAV and challenged with live ISAV.

FIG. 2 is a digital image of the results of SDS-PAGE analysis of purified ISAV proteins.

FIG. 3 is a graph illustrating the results of a humoral immune response to whole killed ISAV in Atlantic salmon.

FIG. 4 is the amino acid sequence alignment of the RNA binding domain of NP from influenza virus A and B with the putative NP RNA binding domain from ISA virus. This alignment was predicted using the Clustal W system.

FIG. 5 is a graph illustrating the titration of ISAV-specific antibodies from Atlantic salmon infected with ISAV.

FIG. 6 is a graph illustrating the ISAV-specific antibodies in sera obtained from Atlantic salmon infected with ISAV or rainbow trout injected with a nucleic acid encoding an ISAV-specific protein.

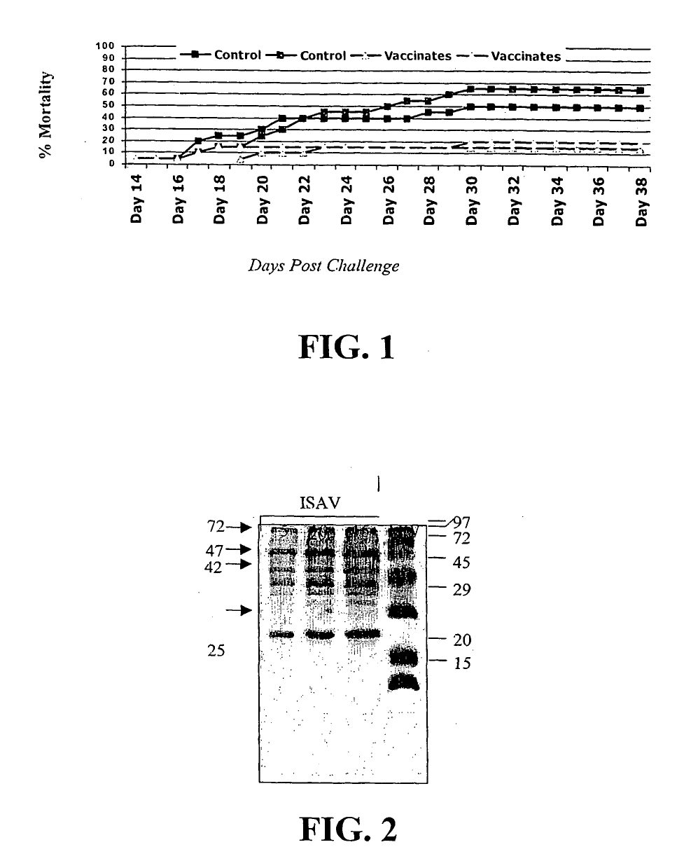

### BRIEF DESCRIPTION OF THE SEQUENCE LISTING

The nucleic acid sequences listed herein are shown using standard letter abbreviations for nucleotide bases. Only one strand of each nucleic acid sequence is shown, but the complementary strand is understood as included by any reference to the displayed strand.

SEQ ID NO: 1 shows a 2.4 kbp nucleic acid fragment of ISAV (segment 1) with a partial open reading frame (orf) encoding the P1 protein.

SEQ ID NO: 2 shows the partial amino acid sequence of the P1 protein encoded by SEQ ID NO: 1.

SEQ ID NO: 3 shows a 2.4 kbp nucleic acid fragment of ISAV (segment 2) with a 2127 bp orf encoding the PB1 protein.

SEQ ID NO: 4 shows the amino acid sequence of the PB1 protein, measuring 709 aa, encoded by SEQ ID NO: 3.

SEQ ID NO: 5 shows a 2.2 kbp nucleic acid fragment of ISAV (segment 3) with a 1851 bp orf encoding the NP protein.

SEQ ID NO: 6 shows the amino acid sequence of the NP protein, measuring 617 aa, encoded by SEQ ID NO: 5.

SEQ ID NO: 7 shows a 1.9 kbp nucleic acid fragment of ISAV (segment 4) with a 1737 bp orf encoding the P2 protein.

SEQ ID NO: 8 shows the amino acid sequence of the P2 protein, measuring 579 aa, encoded by SEQ ID NO: 8.

SEQ ID NO: 9 shows a 1.6 kbp nucleic acid fragment of ISAV (segment 5) with a 1335 bp orf encoding the P3 protein.

SEQ ID NO: 10 shows the amino acid sequence of the P3 protein, measuring 445 aa, encoded by SEQ ID NO: 9.

SEQ ID NO: 11 shows a 1.5 kbp nucleic acid fragment of ISAV (segment 6) with an 1185 bp orf encoding the HA protein.

SEQ ID NO: 12 shows the amino acid sequence of the HA protein, measuring 395 aa, encoded by SEQ ID NO: 10.

SEQ ID NO: 13 shows a 1.3 kbp nucleic acid fragment of ISAV (segment 7) with a 771 bp orf encoding the P4 protein and a 441 bp orf encoding the P5 protein.

SEQ ID NO: 14 shows the amino acid sequence of the P4 protein, measuring 257 aa, encoded by SEQ ID NO: 13.

SEQ ID NO: 15 shows the amino acid sequence of the P5 protein, measuring 147 aa, also encoded by SEQ ID NO: 13.

SEQ ID NO: 16 shows a 1.0 kbp nucleic acid fragment of ISAV (segment 8) with a 705 bp orf encoding the P6 protein and a 552 bp orf encoding the P7 protein.

SEQ ID NO: 17 shows the amino acid sequence of the P6 protein, measuring 235 aa, encoded by SEQ ID NO: 16.

SEQ ID NO: 18 shows the amino acid sequence of the P7 protein, measuring 184 aa, also encoded by SEQ ID NO: 16.

### DETIALED DESCRIPTION

Abbreviations

aa=ammo acid

bp=base pair

ISA=infectious salmon anemia

ISAV=infectious salmon anemia virus

kbp=kilo-base pair

orf=open reading frame

PCR=polymerase chain reaction

RT=reverse transcription

Terms

The following explanations of terms are provided in order to facilitate review of the embodiments described herein. Explanations of common terms also can be found in Rieger et al., Glossary of Genetics: Classical and Molecular, 5th edition, Springer-Verlag: New York, 1991; Lewin, Nucleic acids VII, Oxford University Press: New York, 1999; and Dictionary of Bioscience, Mcgraw-Hill: New York, 1997.

The singular forms “a,” “an,” and “the” refer to one or more than one, unless the context clearly dictates otherwise. For example, the term “comprising a nucleic acid” includes single or plural nucleic acids and is considered equivalent to the phrase “comprising at least one nucleic acid.”

The term “or” refers to a single element of stated alternative elements or a combination of two or more elements. For example, the phrase “a first nucleic acid or a second nucleic acid” refers to the first nucleic acid, the second nucleic acid, or both the first and second nucleic acids.

As used herein, “comprises” means “includes.” Thus, “comprising A and B” means “including A and B,” without excluding additional elements.

The standard one- and three-letter nomenclature for amino acid residues is used.

Amplification of a Nucleic Acid.

Any of several techniques that increases the number of copies of a nucleic acid molecule. An example of amplification is the polymerase chain reaction (PCR), in which a sample containing the nucleic acid is contacted with a pair of oligonucleotide primers under conditions that allow for the hybridization of the primers to nucleic acid in the sample. The primers are extended under suitable conditions, dissociated from the template, and then re-annealed, extended, and dissociated to amplify the number of copies of the nucleic acid. The amplification products (called “amplicons”) can be further processed, manipulated, or characterized by (without limitation) electrophoresis, restriction endonuclease digestion, hybridization, nucleic acid sequencing, ligation, or other techniques of molecular biology. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in European Patent Appl. 320 308; gap filling ligase chain reaction amplification, as disclosed in U.S. Pat. No. 5,427,930; and NASBA™ RNA transcription-free amplification, as disclosed in U.S. Pat. No. 6,025,134.

Conservative Amino-Acid Substitution.

Conservative amino acid substitutions in a polypeptide, such as an ISAV polypeptide, include those listed in Table 1 below.

TABLE 1

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Non-conservative substitutions are those that disrupt the secondary, tertiary, or quaternary conformation of a polypeptide. Such non-conservative substitutions can result from changes in: (a) the structure of the polypeptide backbone in the area of the substitution; (b) the charge or hydrophobicity of the polypeptide; or (c) the bulk of an amino acid side chain. Substitutions generally expected to produce the greatest changes in polypeptide properties are those in which: (a) a hydrophilic residue is substituted for (or by) a hydrophobic residue; (b) a proline is substituted for (or by) any other residue; or (c) a residue having a bulky side chain, for example, phenylalanine, is substituted for (or by) one not having a side chain, for example, glycine. In particular embodiments, a residue having an electropositive side chain, for example, lysyl, arginyl, or histadyl, is not substituted for (or by) an electronegative residue, for example, glutamyl or aspartyl.

Analog or Homolog.

An analog is a molecule that differs in chemical structure from a parent compound. A homolog differs by an increment in the chemical structure (such as a difference in the length of a nucleic acid or amino acid chain), a molecular fragment, a structure that differs by one or more functional groups, or a change in ionization.

Antigen.

A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. The term “antigen” includes all related antigenic epitopes.

Animal.

A living, multi-cellular, vertebrate organism, including, for example, mammals, birds, reptiles, and fish. The term “aquaculture animal” includes all species suitable for aquaculture farming, such as fish, cephalopods, and crustaceans, including the specific species described herein. Similarly, the term “subject” includes both human and veterinary subjects, such as aquaculture animals.

cDNA (Complementary DNA).

A piece of DNA lacking internal, non-coding segments (introns) and regulatory sequences that determine transcription. cDNA can be synthesized in a laboratory by reverse transcription from messenger RNA extracted from cells.

Complementarity.

A nucleic acid that performs a similar function to the sequence to which it is complementary. The complementary sequence does not have to confer replication competence in the same cell type to be complementary, but merely confer replication competence in some cell type.

Delivery of Compositions.

For administration to animals, purified active compositions can be administered alone or combined with an acceptable carrier. Preparations can contain one type of therapeutic molecule, or can be composed of a combination of several types of therapeutic molecules. The nature of the carrier will depend on the particular mode of administration being utilized. For instance, parenteral formulations usually comprise injectable fluids that include physiologically acceptable fluids such as water, physiological saline, balanced salt solutions, aqueous dextrose, glycerol or the like as a vehicle. For solid compositions (e.g., powder, pill, tablet, or capsule forms), which can be added to an aquaculture environment, conventional non-toxic solid carriers can include, for example, mannitol, lactose, starch, or magnesium stearate. In addition to biologically-neutral carriers, compositions to be administered to fish can contain minor amounts of non-toxic auxiliary substances, such as wetting or emulsifying agents, preservatives, and pH buffering agents and the like, for example sodium acetate or sorbitan monolaurate.

It is also contemplated that the nucleic acids could be delivered to cells subsequently expressed by the host cell, for example through the use viral vectors, plasmid vectors, or liposomes administered to fish.

Compositions of the present invention can be administered by any means that achieve their intended purpose. Amounts and regimens for the administration of the nucleic acids, or an active fragment thereof, can be readily determined.

For use in treating viral infections, compositions are administered in an amount effective to inhibit viral infection or progression of an existing infection, or administered in an amount effective to inhibit or alleviate a corresponding disease. In one embodiment, infection is completely prevented.

Typical amounts initially administered would be those amounts adequate to achieve tissue concentrations at the site of action which have been found to achieve the desired effect in vitro. The compositions can be administered to a host in vivo, for example through systemic administration, such as intravenous, intramuscular, or intraperitoneal administration. The compositions also can be administered intralesionally, through scarification of the skin, intrabuccal administration, cutaneous particle bombardment, or by immersion in water containing a nucleic acid composition described herein (for uptake by the fish). Additionally, the nucleic acid compositions can be administered by encapsulation with a nanoparticle matrix composed of a nucleic acid in methacrylic acid polymer, and an attenuated bacteria (such as Yersinia ruckeri, Edwardsiella ictaluri, Aeromonas salmonicida, or Vibrio anguillarum) carrying the nucleic acid for delivery by immersion administration (see, e.g., U.S. Pat. No. 5,877,159, herein incorporated by reference).

Effective doses for using compositions can vary depending on the severity of the condition to be treated, the age and physiological condition of the fish, mode of administration, and other relevant factors. Thus, the final determination of the appropriate treatment regimen can be made by someone at the site of the fish, such as an operator or employee of an aquaculture facility. Typically, the dose range will be from about 1 μg/kg body weight to about 100 mg/kg body weight, such as about 10 μg/kg body weight to about 900 μg/kg body weight, or from about 50 μg/kg body weight to about 500 μg/kg body weight, or from about 50 μg/kg body weight to about 150 μg/kg body weight, such as about 100 μg/kg body weight. Nanogram quantities of transforming DNA have been shown to be capable of inducing an immune response in fish (see, e.g., Corbeil, S., et al., Vaccine 18(25):2817-24 (2000), herein incorporated by reference).

The dosing schedule can vary from a single dosage to multiple dosages given several times a day, once a day, once every few days, once a week, once a month, annually, biannually, biennially, or any other appropriate periodicity. The dosage schedule can depend on a number of factors, such as the species' or subject's sensitivity to the composition, the type and severity of infection, route of administration, and the volume of the container that contains the fish. In the case of a more aggressive disease, compositions can be administered by alternate routes, including intramuscularly and by environmental uptake. Continuous administration also can be appropriate in some circumstances, for example, immersing fish or other aquaculture animals in water containing the composition.

Hybridization Conditions.

“Stringent conditions” encompass conditions under which hybridization will only occur if there is less than 25% mismatch between the hybridization probe and the target sequence. “Stringent conditions” can be broken down into particular levels of stringency for more precise measurement. Thus, as used herein, “moderate stringency” conditions are those under which DNA molecules with more than 25% sequence variation (also termed “mismatch”) will not hybridize; conditions of “medium stringency” are those under which DNA molecules with more than 15% mismatch will not hybridize, and conditions of “high stringency” are those under which DNA sequences with more than 10% mismatch will not hybridize. Conditions of “very high stringency” are those under which DNA sequences with more than 6% mismatch will not hybridize.

Hybridization.

Oligonucleotides hybridize by hydrogen bonding, which includes Watson-Crick, Hoogsteen, or reversed Hoogsteen hydrogen bonding between complementary nucleotide units. For example, adenine and thymine are complementary nucleotides that pair through formation of hydrogen bonds. “Complementary” refers to sequence complementarity between two nucleotide units. For example, if a nucleotide unit at a certain position of an oligonucleotide is capable of hydrogen bonding with a nucleotide unit at the same position of a nucleic acid molecule, then the oligonucleotides are complementary to each other at that position. The oligonucleotide and the nucleic acid molecule are complemtary to each other when a sufficient number of corresponding positions in each molecule are occupied by nucleotide units that can hydrogen bond with each other.

Nucleic acid molecules and nucleotide sequences derived from the disclosed molecules also can be defined as nucleotide sequences that hybridize under stringent conditions to the sequences disclosed, or fragments thereof.

“Specifically hybridizable” and “complementary” are terms which indicate a sufficient degree of complementarity, such that stable and specific binding occurs between an oligonucleotide and the target nucleic acid. An oligonucleotide need not be 100% complementary to the target to be specifically hybridizable. An oligonucleotide is specifically hybridizable when binding of the oligonucleotide to the target molecule interferes with the normal function of the target and there is a sufficient degree of complementarity to avoid non-specific binding of the oligonucleotide to non-target sequences under conditions in which specific binding is desired (for example, under physiological conditions in the case of in vivo assays) or under conditions in which the assays are performed.

Hybridization conditions resulting in particular degrees of stringency will vary depending upon the nature of the hybridization, method of choice, and the composition and length of the hybridizing nucleic acid used. Generally, the temperature of hybridization and the ionic strength (especially the Na⁺ concentration) of the hybridization buffer will determine the stringency of hybridization. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001).

Epitope.

A site on an antigen at which an antibody can bind, the molecular arrangement of the site determining the combining antibody. A portion of an antigen molecule that determines its capacity to combine with the specific combining site of its corresponding antibody in an antigen-antibody interaction.

Nucleotide Molecules That Hybridize.

Nucleotide molecules and sequences which are derived from the disclosed nucleotide molecules as described above also can be defined as nucleotide sequences that hybridize under stringent conditions to the nucleotide sequences disclosed, or fragments thereof.

Genetic Fragment.

Any nucleic acid derived from a larger nucleic acid.

Heterologous.

Originating from a different organism or distinct tissue culture, such as from a different species or cell line.

Homologs.

Two nucleotide sequences that share a common ancestral sequence and diverged when a species carrying that ancestral sequence split into two species.

Isolated.

An “isolated” biological component (such as a nucleic acid, polypeptide, protein, or organelle) has been substantially separated, produced apart from, or purified away from other biological components (for example, other chromosomal and extrachromosomal DNA and RNA, and polypeptides) found in the cell of the organism in which the component naturally occurs. Nucleic acids, polypeptides, and proteins that have been “isolated” thus include nucleic acids and polypeptides purified by standard purification methods. The term also embraces nucleic acids, polypeptides, and proteins that are chemically synthesized or prepared by recombinant expression in a host cell.

Nucleic Acid.

A deoxyribonucleotide or ribonucleotide polymer in either single or double stranded form. Unless otherwise limited, this term encompasses known analogues of natural nucleotides that hybridize to nucleic acids in a manner similar to naturally occurring nucleotides. An “oligonucleotide” (or “oligo”) is a linear nucleic acid of up to about 250 nucleotide bases in length. For example, a polynucleotide (such as DNA or RNA) which is at least 5 nucleotides long, such as at least 15, 50, 100, or even more than 200 nucleotides long.

Operably Linked.

A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous. Where necessary to join two protein coding regions, the operably linked sequences are in the same reading frame.

Expression Control Sequence.

A nucleic acid sequence that affects, modifies, or influences expression of a second nucleic acid sequence. Promoters, operators, repressors, and enhancers are examples of expression control sequences.

ORF (Open Reading Frame).

A series of nucleotide triplets (codons) coding for amino acids without any termination codons. These sequences are usually translatable into a peptide.

Ortholog.

Two nucleotide sequences are orthologs of each other if they share a common ancestral sequence and diverged when a species carrying that ancestral sequence split into two species. Orthologous sequences are also homologous sequences.

Parenteral.

Administered outside of the intestine and not via the alimentary tract. Generally, parenteral formulations are those that will be administered through any possible mode except ingestion. This term especially refers to injections, whether administered intravenously, intrathecally, intramuscularly, intraperitoneally, or subcutaneously, and various surface applications including intranasal, intradermal, and topical application, for instance.

Polypeptide.

Any chain of amino acids, regardless of length or post-translational modification (for example, glycosylation or phosphorylation).

Polypeptide Sequence Homology.

In certain embodiments, a polypeptide is at least about 70% homologous to a corresponding sequence (such as SEQ ID NO:1) or a native polypeptide (such as HA), such as at least about 80% homologous, and even at least about 95% homologous. Such homology is considered to be “substantial homology.”

Polypeptide homology is typically analyzed using sequence analysis software, such as the programs available from the Genetics Computer Group (Madison, Wis., see the Genetics Computer Group website)

Portion of a Nucleic Acid Sequence.

At least 10, 20, 30, 40, 50, 60, 70, 80, or more contiguous nucleotides of the relevant sequence.

Promoter.

A promoter is one type of expression control sequence composed from an array of nucleic acid sequences that directs transcription of a nucleic acid. A promoter includes necessary nucleic acid sequences near the start site of transcription, such as a TATA element. A promoter also can include distal enhancer or repressor elements that can be located as much as several thousand base pairs from the start site of transcription. A promoter can be constitutive or inducible. An inducible promoter directs transcription of a nucleic acid operably coupled to it only under certain environmental conditions, such as in the presence of metal ions or above a certain temperature.

Protein Purification.

Polypeptides can be purified by any method known to one of skill in the art. Exemplary, non-limiting methods are described in: Guide to Protein Purification: Methods Enzymologyl, ed. Deutscher, Academic Press, San Diego, 1997; and Scopes, Protein Purification: Principles and Practice, 3^{rd} ed., Springer Verlag, New York, 1994.

Purified.

The term purified does not require absolute purity; rather, it is intended as a relative term. Thus, for example, a purified nucleic acid is one in which the nucleic acid is more enriched than the nucleic acid is in its natural environment within a cell. In one embodiment, a preparation is purified if a component, such as a nucleic acid, represents at least 50% of the total amount of that component (e.g. the nucleic acid content) of the preparation.

Recombinant.

A recombinant nucleic acid is one that has a sequence that is not naturally occurring, or has a sequence that is made by an artificial combination of two otherwise separated segments of sequence. This artificial combination can be accomplished by chemical synthesis or artificial manipulation of isolated segments of nucleic acids, for example by genetic engineering techniques. Similarly, a recombinant protein is one encoded for by a recombinant nucleic acid molecule. The term recombinant includes nucleic acids that have been altered solely by deletion of a portion of the nucleic acid.

Resistance to Infection.

Animnals resistant to infection will demonstrate decreased symptoms of infection compared to non-resistant animals. Evidence of resistance to infection can appear as, for example, lower rates of mortality; increased life-spans measured after exposure to the infective agent; fewer or less intense physiological symptoms, such as fewer lesions; or decreased cellular or tissue concentrations of the infective agent. In one embodiment, resistance to infection is demonstrated by a heightened immune response.

Sequence Identity.

The similarity between two nucleic acid sequences, or two amino acid sequences, is expressed in terms of the similarity between the sequences, otherwise referred to as sequence identity. Sequence identity is frequently measured in terms of percentage identity (or similarity or homlogy); the higher the percentage, the more similar are the two sequences.

Methods of alignment of sequences for comparison are well-known in the art. Various programs and alignment algorithms are described in: Smith and Waterman, Adv. Appl. Math. 2:482, 1981; Needleman and Wunsch, J. Mol. Bio. 48:443, 1970; Pearson and Lipman, Methods in Molec. Biology 24: 307-331, 1988; Higgins and Sharp, Gene 73:237-244, 1988; Higgins and Sharp, CABIOS 5:151-153, 1989; Corpet et al., Nucleic Acids Research 16:10881-90, 1988; Huang et al., Computer Applications in BioSciences 8:155-65,1992; and Pearson et al., Methods in Molecular Biology 24:307-31,1994. Altschul et al. (1994) presents a detailed consideration of sequence alignment methods and homology calculations.

The NCBI Basic Local Alignment Search Tool (BLAST) (Altschul et al., J. Mol. Biol. 215:403-410, 1990) is available from several sources, including the National Center for Biological Information (NBCI, Bethesda, Md.) and on the Internet, for use in connection with the sequence analysis programs blastp, blasm, blastx, tblastn and tblastx. It can be accessed at the NCBI website.

Homologs of the nucleic acids and polypeptides described herein are typically characterized by possession of at least 70% sequence identity counted over the full length alignment with a disclosed sequence using the NCBI Blast 2.0, gapped blastp set to default parameters. Such homologous nucleic acids or peptides will possess at least 70%, at least 80%, or even at least 90% or 95% sequence identity determined by this method. When less than the entire sequence is being compared for sequence identity, homologs will possess at least 70%, such as at least 85%, or even at least 90% or 95% sequence identity over short windows of 10-20 amino acids. Methods for determining sequence identity over such short windows are described at the NCBI website. These sequence identity ranges are provided for guidance only; it is entirely possible that strongly significant homologs or other variants could be obtained that fall outside of the ranges provided.

In addition to the peptide homologs described above, nucleic acid molecules that encode such homologs are encompassed by alternative embodiments. One indication that two nucleic acid sequences are substantially identical is that the polypeptide which the first nucleic acid encodes is immunologically cross reactive with the polypeptide encoded by the second nucleic acid. Another indication that two nucleic acid sequences are substantially identical is that the two molecules hybridize to each other under stringent conditions. Stringent conditions, as described above, are sequence dependent and are different under different environmental parameters.

Nucleic acid sequences that do not show a high degree of identity can nevertheless encode similar amino acid sequences, due to the degeneracy of the genetic code. It is understood that changes in nucleic acid sequence can be made using this degeneracy to produce multiple nucleic acid sequence that all encode substantially the same polypeptide.

Nucleic cid molecules demonstrating substantial similarity may be of different types. A DNA molecule can demonstrate some degree of identity to an RNA molecule by comparing the sequences, where a T residue on the DNA molecule is considered identical to a U residue on the RNA molecule.

Substantially Similar.

When optimally aligned (with appropriate nucleotide insertions or deletions) with the other nucleic acid (or its complementary strand), there is nucleotide sequence identity in at least about 50%, 60%, 70%, 80% or 90 to 95% of the nucleotide bases.

Therapeutic Agent.

Includes treating agents, prophylactic agents, and replacement agents made from nucleic acid and/or amino acid compositions described herein.

Therapeutically Effective Amount or Effective Amount.

A quantity sufficient to achieve a desired effect in situ, in vitro, in vivo, or within a subject being treated. For instance, the effective amount can be the amount necessary to inhibit viral proliferation or to measurably alter progression of disease. In general, this amount will be sufficient to measurably inhibit virus (ISAV) replication or infectivity.

An effective amount can be administered in a single dose, or in several doses, for example daily, during a course of treatment. However, the effective amount can depend on the composition applied or administered, the subject being treated, the severity and type of the affliction, and the manner of administration.

The compositions disclosed have application in various settings, such as aquaculture, environmental containment, or veterinary settings. Therefore, the general term “subject being treated” is understood to include all fish that are or may be infected with a virus or other disease-causing microorganism that is susceptible to neutralization by the compositions described herein.

Transduced, Transformed, and Transfected.

A virus or vector “transduces” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. Transfection is the uptake by eukaryotic cells of a nucleic acid from the local environment and can be considered the eukaryotic counterpart to bacterial transformation.

As used herein, the term transformation encompasses all techniques by which a nucleic acid molecule might be introduced into a cell.

Transgene.

An exogenous gene supplied by a vector.

Transgenic.

Of, pertaining to, or containing a gene, ORF, or other nucleic acid native to another species, microorganism, or virus. The term “transgenic” includes transient and permanent transformation, where the nucleic acid integrates into chromosomal DNA, including the germ line, or is maintained extrachromosomally.

Variants of Amino Acid and Nucleic Acid Sequences.

The production of proteins disclosed herein (for example, HA) can be accomplished in a variety of ways. DNA sequences which encode for the protein, or a fragment of the protein, can be engineered such that they allow the protein to be expressed in eukaryotic cells, bacteria, insects, and/or plants. In order to accomplish this expression, the DNA sequence can be altered and operably linked to other regulatory sequences. The final product, which contains the regulatory sequences and the nucleic acid, is referred to as a vector. This vector can then be introduced into the eukaryotic cells, bacteria, insect, and/or plant. Once inside the cell, the vector allows the protein to be produced.

The DNA can be altered in numerous ways without affecting the biological activity of the encoded protein. For example, PCR can be used to produce variations in the DNA sequence which encodes an ISAV peptide. Such variants can be variants that are optimized for codon preference in a host cell that is to be used to express the protein, or other sequence changes that facilitate expression.

At least two types of cDNA sequence variant can be produced. In the first type, the variation in the cDNA sequence is not manifested as a change in the amino acid sequence of the encoded polypeptide. These silent variations are simply a reflection of the degeneracy of the genetic code. In the second type, the cDNA sequence variation does result in a change in the amino acid sequence of the encoded protein. In such cases, the variant cDNA sequence produces a variant polypeptide sequence. In order to preserve the functional and immunologic identity of the encoded polypeptide, certain embodiments utilize amino acid substitutions that are conservative.

Variations in the cDNA sequence that result in amino acid changes, whether conservative or not, can be minimized in order to preserve the functional and immunologic identity of the encoded protein. Variant amino acid sequences can, for example, be 70, 80%, 90%, or even 95% identical to the native amino acid sequence.

Vector.

A nucleic acid molecule as introduced into a host cell, thereby producing a transformed host cell. A vector can include nucleic acid sequences that permit it to replicate in the host cell, such as an origin of replication. A vector can also include one or more therapeutic genes and/or selectable marker genes and other genetic elements. A vector can transduce, transform or transfect a cell, thereby causing the cell to express nucleic acids and/or proteins other than those native to the cell. A vector optionally includes materials to aid in achieving entry of the nucleic acid into the cell, such as a viral particle, liposome, protein coating, or the like. Plasmids are often used as vectors to transform fish cells.

ISAV Specific Nucleic Acids and Polypeptides

Polypeptides and nucleic acid molecules are disclosed herein, as are and treatments for protecting fish, shellfish, and other aquacultured organisms against ISAV. The nucleic acids include segments of the ISAV genome, such as the segments described herein and summarized in Table 5 below, or fragments thereof. Also included are fragments of the ISAV genome that overlap the individual segments summarized in Table 5.

ISAV polypeptides are described herein, as are nucleic acids that encode the ISAV polypeptides. ISAV polypeptides include, but are not limited to, P1, PB1, (nucleotprotein) NP, P2, P3, hemaglutinin (HA), P4, P5, P6, and P7. Thus, polypeptides having a sequence as set forth as SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 15, SEQ ID NO: 17, or SEQ ID NO: 18, an antigenic fragment thereof, or a conservative variant thereof, are provided herein.

Polypeptides can be divided into sections, such as an N-terminal and a C-terminal portion. Thus, in one embodiment, polypeptide fragments are provided that include the N-terminal or the C-terminal portion of an ISAV polypeptide.

Antigenic fragments of an ISAV polypeptide are provided herein. An antigenic fragment is any ISAV polypeptide that can produce an immune response in fish. The immune response can be a B cell or a T cell response, or induction of a cytokine.

Also provided herein are nucleic acids that encode and ISAV polypeptide. In one embodiment, a nucleic acid is provided that encodes a P1 polypeptide. One specific non-limiting example of a P1 polypeptide is the sequence set forth as SEQ ID NO:2, a fragment, or a conservative variant thereof.

In another embodiment, a nucleic acid is provided that encodes a hemaglutinin (HA) polypeptide. One specific, non-limiting example of an HA polypeptide is the sequence as set forth as SEQ ID NO:12, a fragment, or a conservative variant thereof.

In a further embodiment, a nucleic acid is provided that encodes a PB1 polypeptide. One specific, non-limiting example of an PB1 polypeptide is the sequence as set forth as SEQ ID NO:4, a fragment, or a conservative variant thereof.

Nucleic acids are also disclosed herein that are substantially similar to particular segments, such as nucleic acids that are at least 70% identical to SEQ ID NO: 1, at least 85% identical to SEQ ID NO: 3, or at least 85% identical to SEQ ID NO: 11. Thus, in one embodiment, a nucleic acids is provided that is are at least 75% identical to SEQ ID NO: 1, at least at least 80% identical to SEQ ID NO: 1, at least 85% identical to SEQ ID NO: 1, at least 90% identical to SEQ ID NO: 1, or at least 95% identical to SEQ ID NO: 1. In another embodiment, a nucleic acid is provided that is at least 90% identical to SEQ ID NO: 3, at least 95% identical to SEQ ID NO: 3, or at least 99% identical to SEQ ID NO:3. In a further embodiment, a nucleic acid is provided that is at least 90% identical to SEQ ID NO: 11, at least 95% identical to SEQ ID NO: 11, or at least 99% identical to SEQ ID NO:11.

In yet another embodiment, nucleic acids are provided that consist essentially of an ISAV nucleic acid sequences, such as a nucleic acid having a sequence as set forth as SEQ ID NO: 1, SEQ ID NO: 3, SEQ ID NO: 5, SEQ ID NO: 7, SEQ ID NO: 9, SEQ ID NO: 11, or SEQ ID NO: 16.

The nucleic acids disclosed herein can be operably linked to a heterologous nucleic acid, such as an expression control sequence. In one embodiment, the expression control sequence is a promoter, such as an interferon response element, beta-actin, a cytokine promoter, a cytomegalovirus promoter, or a fish viral promoter. In particular embodiments, the promoter is an inducible promoter, such as a heat shock promoter, or a promoter induced by a hormone or a metal ion. Nucleic acid compositions can contain other elements, such as additional expression control elements, structural sequences, origins of replication, or multiple coding sequences. In particular embodiments, an expression control sequence operably linked to a nucleic acid encoding an ISAV polypeptide is included in a vector, including, but not limited to, a plasmid, a viral vector, a phagemid, or a cosmid. Cloning vectors include, but are not limited to, those described in U.S. Pat. No. 5,998,697. Viral vectors include, but are not limited to, retroviral or adenoviral vectors.

The nucleic acid compositions described herein can be utilized in vitro, in vivo, or in situ. For example, a nucleic acid at least 70% identical to SEQ ID NO: 1 could be used to study an antigenic epitope of interest for in vitro production and manipulation, or to study its effect on cell physiology or activity in vivo, or for tissue-specific expression analysis in situ. Particular uses of these nucleic acid compositions also are illustrated in the Examples below.

In some embodiments, the nucleic acid molecule encodes an antigenic sequence, such as an antigenic sequence for pathogens of aquacultural animals. Aquacultural animals include fish (both bony and cartilaginous fish), shellfish and other arthropods, and molluscs. Particular exemplary aquicultural animals include, but are not limited, to the following: salmonids, such as rainbow trout (Oncorhynchus mykiss), coho salmon (O. kisutch), chinook salmon (O. tshawytcha), amago salmon (O. rhodurus), chum salmon (O. keta Walbaum), sockeye salmon (O. nerka), Atlantic salmon (Salmo salar), arctic char (Salvelinus alpinus), brown trout (Salmo trutta), cutthroat trout (Salmo clarkii), and brook trout (Salvelinus fontinalis); catfish (Ictalurus punctatus); tilapia (Oreochromis niloticusand and Oreochromis mozambicus); sea bream (Archosargus rhomboidalis), seabass (Dicentrarchus labrax); flounder (Paralichthys dentatus); sturgeon (Scaphirhynchus albus); eels (including members of the order Anguilliformes, class Actinopterygii, such as Conger spp., Ariosoma spp., Gnathophis spp., Coloconger spp., Anguilla spp., Nessorhamphus spp., Cynoponticus spp., Anarchias spp., Echidna spp., Enchelycore spp., Gymnothorax spp., and Uropterygius spp.); cephalopods (octopi and squids); crustaceans (including lobsters, prawns, shrimp, crabs, and crayfish in the order Decapoda); and bivalves (clams and oysters, such as Ostrea edulis and Pisidium spp.). In some embodiments, the aquaculture animal is a fish, such as a salmonid. In particular embodiments, In some embodiments, the nucleic acid molecule encodes a polypeptide that is an antigenic sequence, such that upon introduction in fish, an immune response is induced against ISAV.

Eliciting an Immune Response in Fish

Some embodiments employ nucleic acid compositions containing nucleic acid sequences encoding antigenic epitopes. In such embodiments, the nucleic acid composition includes an expression control sequence operably linked to a nucleic acid sequence encoding an antigenic epitope, thus driving expression of the nucleic acid sequence and eliciting an immune response to the antigenic epitope in the fish. In particular embodiments, the antigen expressed is a polypeptide encoded by ISAV, which elicits an immune response in the fish against ISAV.

In any such embodiment, the fish utilized can belong to a particular species, such as rainbow trout, coho salmon, chinook salmon, amago salmon, chum salmon, sockeye salmon, Atlantic salmon, arctic char, brown trout, cutthroat trout, brook trout, catfish, tilapia, sea bream, seabass, flounder, and sturgeon.

Exemplary, non-limiting uses of these nucleic acid compositions are described in the Examples below.

In certain embodiments, any of the nucleic acid compositions described herein is used to transform fish tissue to produce a transgenic fish. In such embodiments, a nucleated cell of the transgenic fish is transformed with a nucleic acid sequence substantially similar to the nucleic acid sequences described herein (for example, SEQ ID NOS.: 1, 3, 5, 7, 9, 11, 13, and 16). In particular embodiments, the nucleic acid is at least 70% identical to SEQ ID NO: 1, at least 85% identical to SEQ ID NO: 3, or at least 85% identical to SEQ ID NO: 11, operably linked to a heterologous nucleic acid sequence.

If it encodes an antigenic epitope, expression of the nucleic acid sequence can induce an immune response to the antigenic epitope within the fish or other aquaculture animal. In such embodiments, the animal exhibits an increased resistence to infection by ISAV as compared to a non-transformed animal of the same species.

In alternative embodiments, the animal subject is treated with a polypeptide composition that functions as an antigenic epitope and induces an immune response within that subject, such as the polypeptides a sequence as set forth as SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 15, SEQ ID NO: 17, or SEQ ID NO: 18, an antigenic fragment thereof, or a conservative variant thereof. In some embodiments, the antigenic polypeptide is a fusion protein, such as a polypeptide as set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 15, SEQ ID NO: 17, or SEQ ID NO: 18, an antigenic fragment thereof, or a conservative variant thereof, coupled to a fusion partner. Such fusion partners include, but are not limited to, antigens from other fish viruses (for example, glycoprotein from rhabdovirus, birnavirus, reovirus, nodavirus, herpes virus, or infectious pancreatic necrosis virus), ISS DNA elements, or T-cell epitopes.

The antigenic polypeptides can be obtained by recombinant methods, such as expression in eukaryotic or bacterial cell culture, or can be chemically synthesized. In particular embodiments, the antigenic polypeptides are recombinantly expressed in a non-mammalian eukaryotic cell culture, such as a fish cell culture, for example a CHSE-214, TO, SHK, RTG-2, or EPC cell culture. Thus, an antigenic polypeptide can be prepared by transforming fish cells with a nucleic acid vector encoding an antigenic polypeptide (including one that is a fusion protein), as described above, culturing the host cells under conditions suitable for expressing the antigenic polypeptide, and then recovering the antigenic polypeptide from the cell culture. Additionally, such cell cultures can be transformed with multiple nucleic acid vectors, thus expressing multiple antigenic polypeptides.

Recovered antigenic polypeptides can then be purified and readied for delivery to the subject (as described above), and the antigenic polypeptide can be combined with a pharmaceutically acceptable salt, carrier, adjuvant, or diluent, and/or other active or inactive ingredients, to form a pharmaceutical composition. The amount or concentration of the antigenic polypeptide within the pharmaceutical composition can vary according to factors such as the effectiveness of the antigenic polypeptide in inducing an immune response within the species of the subject, the severity of the disease or condition to be treated, the route or frequency of administration, or other relevant factors. These compositions also can be tested for immunogenicity prior to delivery to a subject using an in vitro assay, such as one of the assays described in the Examples below.

Once prepared, an effective amount of the antigenic polypeptide or pharmaceutical composition is delivered to the subject via a suitable route of administration, for example, intramuscular, intraperitoneal, oral, immersion, or ultrasound administration. An effective amount is any amount that enhances the immunocompetence of the subject treated and elicits some immunity against ISAV, for example, by delaying, inhibiting, or even preventing the onset or progression of ISA. In some embodiments, the subject's immune system is stimulated by at least about 15%, such as by at least about 50%, or even at least about 90%.

### EXAMPLES

The following examples are intended to illustrate the invention, but not to limit it in any manner, either explicitly or implicitly. While these examples are typical of those that might be used, other procedures, methodologies, or techniques known to those skilled in the art alternatively can be used.

### Example 1

### Vaccine Trial to Test the Efficacy of the Whole Killed ISAV Vaccine

Atlantic salmon, each weighing about 90 g, were anaesthetized and intraperitoneal injected with 0.2 ml of a solution of whole killed ISAV. Four groups of twenty fish in each group were studied; two groups were injected with whole killed ISAV and two groups were injected with an equivalent amount of saline.

Following vaccination, the salmon were acclimated to saltwater at 12° C. and held for 798 degree days prior to challenge with ISAV. Twenty-four native Atlantic salmon were anaesthetized, fin clipped for identification, and intraperitoneal injected with 1 ml ISAV infected CHSE-214 cell culture supernatant (1×10⁷ TCID₅₀/ml). Six of these fish were added to each of the four tanks containing the groups of either the vaccinated or control fish. Saline-injected control salmon experienced a cumulative mortality of 57.5% when challenged with ISAV by cohabitation. Vaccinated salmon had a cumulative mortality of 17.5%. The RPS value of the ISAV whole killed vaccine was 70.0%.

### Example 2

### Humoral Immune Response to Whole Killed ISAV

Atlantic salmon were vaccinated with the two different serials of whole killed ISAV in MV4 at two different antigen doses, and sera was collected at 350 degree-days, 696 degree-days, and 972 degree-days after vaccination.

ISAV-specific antibodies in the sera were detected by enzyme-linked immunosorbent assay (ELISA). ISAV antigen was dried onto wells of an ELISA plate overnight at 37° C. Wells were rinsed three times with PBS/Tween and, serial dilutions of anti-ISAV Atlantic salmon sera were added in triplicate. After one hour, wells were rinsed three times with PBS/Tween. A second antibody, mouse anti-salmon immunoglobulin, was added to each well, the plates were incubated for one hour, and then rinsed with PBS/Tween in triplicate. After incubation with a third antibody, goat anti-mouse IgG conjugated to alkaline phosphatase, the wells were washed with PBS/Tween and developer containing p-nitrophenyl phosphate was added. The absorbance was measured at 405 nm.

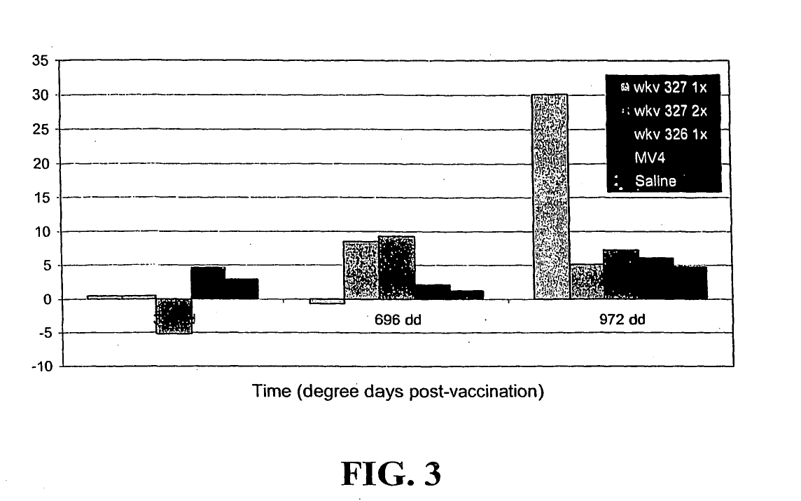

FIG. 3 illustrates the results of this humoral response trial. Antibody levels were reported as a percent of the value obtained with mAb 10A3 to normalize the variation between ELISA plates. A 1× dose of the whole killed ISAV vaccine serial 327 elicits a humoral immune response at 972 degree-days post-vaccination.

### Example 3

### Virus and RNA Purification

Virus was prepared by inoculating CHSE-214 cell monolayers in a 6300 cm² Cell Factory® (Nalge Nunc International, Rochester, N.Y., USA) with ISA virus. Following complete cell lysis, the cell culture supernatant was harvested from the Cell Factory® as disclosed by the manufacturer and filtered through a sterile 0.45 micron filter to remove extraneous cell debris. After dialysis against solid polyethylene glycol to reduce the volume, the cell culture supernatant was centrifuged for two hours at 24,000 rpm using a SW28 rotor and a Beckman L8-70M ultracentrifuge (Beckman Coulter, Inc., Fullerton, Calif., USA). The pelleted virus was resuspended in TNE, layered on a 25, 35 and 45% sucrose gradient and centrifuged for 3 hours at 27,000 rpm using a SW28 rotor and a Beckman L8-70M ultracentrifuge. Virus at the interface of the 35 and 45% sucrose layers was collected, resuspended in TNE and centriged for two hours at 24,000 rpm using a SW28 rotor and a Beckman L8-70M ultracentrifuge. The fraction collected from the 35-45% interface was enriched with material that reacted with an ISAV-specific monoclonal antibody. Viral RNA was isolated from the pelleted virus using Trizol (Gibco) as described by the manufacturer and then used to construct cDNA libraries.

Purified ISAV was resuspended in SDS-sample buffer. The solubilized proteins were separated by SDS-PAGE on a 5% stacking gel and a 12% resolving gel and visualized by Coomassie blue staining. As shown in FIG. 2, after SDS-PAGE, four distinct protein bands were evident: 72 kDa, 47 kDa, 42 kDa, 25 kDa.

Seven proteins from purified ISAV were subjected to N-terminal amino acid sequence analysis. The proteins of purified ISAV were separated by SDS-PAGE, blotted onto PVDF membrane (BioRad Laboratories, Hercules, Calif.) and stained with 0.1% Coomassie blue R-250 in 40% methanol/1% acetic acid. The stained protein bands were cut out of the membrane and subjected to N-terminal amino acid sequence analysis using an Applied Biosystems model 470A gas-phase sequencer (Applied Biosystems, Inc., Foster City, Calif.) or an Applied Biosystems model 473 liquid-phase sequencer with on-line phenylthiohydantoin analysis. The results of this sequencing analysis are shown in Table 2.

TABLE 2
N-terminal amino acid sequence analysis of ISAV proteins

| Protein | | |
|---|---|---|
| MW | | Similarity |
| (kDa) | Sequence analysis | analysis |
| 25 | KVSFDMA; SLQGPVA (internal | No similarity found |
| | sequence) | |
| 35 | N-terminally blocked | N/A |
| 38 | N-terminally blocked | N/A |
| 40 | RLXLRNHPDTTWIGDSRSDQSRXNQ | Putative segment |
| | (N-terminal sequence) | 7 ISAV; segment 4 |
| | | Influenza C |
| 42 | RLXLRNHPDTTWIGDSRSDQSRXNQ | HA (segment 6) |
| | (N-terminal sequence) | |
| 47 | EPXIXENPTXLAI (N-terminal sequence) | 5: E-7 (segment 5) |
| 72 | N-terminally blocked | N/A |

### Example 4

### Construction of cDNA Libraries

Strategies for Cloning the ISA V Genome.

Approach 1: First strand cDNA was synthesized from ISA vRNA by reverse transcription with the ISAV-specific primer (SEQ ID NO: 19):

5′-AAGCAGTGGTAACAACGCAGAGTAGCAAAGA-3′

RNA (100 ng) isolated from purified ISAV or CHSE-214 cells (control) was mixed with ISAV primer (20 pmol/μl), incubated at 80° C. for 5 min and then combined with the following in a total of 20 μl: 4 μA 5× first strand buffer (Gibco Invitrogen Corp., Carlsbad, Calif.), 2 μl 10 mM dNTP mix (Boehringer Mannheim), 1 μl 0.1 M DTT (Gibco) and 1 μl Superscript II reverse transcriptase (15 U/μl; Gibco). The mixture was incubated at 25° C. for 10 min and then at 42° C. for 1 hr.

The first strand ISAV cDNA products synthesized by reverse transcription were PCR amplified using the ISAV primer and random hexamers. To the first strand reaction, the following components were added in a total of 100 μl: 1.5 μl 10 mM DNTP mix (Boehringer Mannheim, 1.25 μl ISAV primer (20 pmol/μl), 1 μl random hexamers (25 pmol/μl; Gibco), 10 μl 10× PCR buffer with Mg²⁺ (Boehringer Mannheim), 1 μl Taq (5 U/μl; Boehringer Mannheim). After 35 cycles of 94° C. for 30 sec, 59° C. for 45 sec and 72° C. for 1 min, the PCR products were extended for 10 min at 72° C. The amplified cDNA products were separated by agarose gel electrophoresis, gel purified and then cloned into the pGEM-T vector as described by the manufacturer (Promega).

Approach 2: First strand cDNA was synthesized from ISA vRNA by reverse transcription with random hexamer primers. RNA (100 ng) isolated from purified ISAV or CHSE-214 cells (control) was mixed with random hexamers (50 ng/μl; Gibco), incubated at 65° C. for 5 min, placed on ice for 2 min and then combined with the following in a total of 20 μl: 4 μl 5× first, strand buffer (Gibco), 2 μl 10 mM dNTP mix (Boehringer Mannheim), 1 μl 0.1 M DTT (Gibco) and 1 μl Superscript II reverse transcriptase (15 U/μl; Gibco). The mixture was incubated at 25° C. for 10 min and then at 50° C. for 50 min.

The TimeSaver cDNA synthesis kit (Pharmacia) was used for second strand cDNA synthesis. The first strand reaction was added to the second strand reaction mix, incubated at 12° C. for 30 min and then at 22° C. for 1 hr. After spin column purification, the blunt ended, double stranded cDNAs were cloned into dephosphorylated, SmaI digested pUC18 (Pharmacia) as outlined by the manufacturer.

For both libraries, E. coli DH5α (Gibco) was transformed with the ligation reactions and the ampicillin-resistant colonies containing either pGEM-T or pUC18 with cloned ISAV cDNA were selected by blue/white screening. The white colonies were transferred to 96 well plates containing 200 μl LB/ampicillin (250 μg/ml)/15% glycerol per well, grown overnight at 37° C. and stored at −20° C.

RT-PCR Amplification of Segments 2, 6 and 8 from ISA V CCBB.

First strand cDNAs for segments 2, 6 and 8 were synthesized from ISA virus RNA by reverse transcription using primers outlined in Table 3 and conditions described above. PCR amplification was used for second strand cDNA synthesis; after 30 cycles of 95° C. for 1 min, 50° C. for 1 min and 72° C. for 2 min, the PCR products were extended for 10 min at 72° C. (see Table 3 for primers). RT-PCR products were gel purified as described by the manufacturer (Qiagen). Table 3. RT and PCR oligonucleotide DNA primers for RNA segments 2, 6 and 8 of ISA virus isolate CCBB.

!Segment? Primer name? Primer sequence (5′-3′)

| | | |
|---|---|---|
| 2 | seg 2-5′F-mRNA | GAACGCTCTTTAATAACCATG |
| | seg 2-3′R-mRNA | TCAAACATGCTTTTTCTTC |
| 6 | HA forward | AGCAAAGATGGCACGATTC |
| | HA reverse | TGCACTTTTCTGTAAACGTACAAC |
| 8 | seg 8-5′F-mRNA | AAGCAGTGGTAACAACGCAGAGTCTATCTACCATG |
| | seg 8-3′R-mRNA | TTATTGTACAGAGTCTTCC |

Selection and Identification of ISAV Clones from the cDNA Libraries.

The contents of one 96-well plate were transferred to one Hybond N⁺ membrane (Amersham) then placed on top of an LB agar plate containing ampicillin (250 μg/ml). Clones were grown on the filters at 37° C. overnight and the filters were processed on soaking pads saturated with the following solutions: 0.5 N NaOH (7 min); 1 M Tris-HCl pH 7.4 (2 min); 1 M Tris-HCl pH 7.4 (2 min); 0.5 M Tris-HCl pH 7.4, 1.5 M NaCl (4 min). The filters were transferred to a bath of 2×SSC (1×SSC is 0.15 M NaCl, 0.015 M Na₃ citrate), 1% sodium dodecyl sulfate (SDS) and then soaked in 2×SSC. After a brief wash in chloroform, the filters were air dried and then baked at 80° C. for 2 hrs. Prehybridization of the filters for 2 hr in 6×SSC, 0.5% SDS, 5× Denhardt's and 0.1 mg/ml E. coli tRNA (Sigma) was followed by hybridization with a probe labelled with [α³²P] dCTP by nick translation. Nick translation was done as outlined by the manufacturer (Amersham). The libraries were initially screened using gel purified, RT-PCR amplified cDNA for segments 2, 6 or 8 of ISAV isolate CCBB. The remaining segments were identified using probes consisting of gel purified, restriction enzyme fragments digested from the plasmids of randomly selected library clones. Library clones were grouped based on the probe to which they hybridized (see Table 4). Eight distinct cDNA hybridization groups were identified. Of these, two groups were found in cDNA library 1 and all but one segment of the ISAV genome in cDNA library 2 (see Table 4).

Plasmid DNA isolated from representative clones of each group using Qiaprep columns (Qiagen) was sequenced at the University of Maine Core Sequencing Facility. Only those sequences that matched other orthomyxovirus sequences or that did not match non-viral sequences were analyzed further.

TABLE 4
Summary of groups formed from screening ISA virus cDNA libraries.

| | | Number of positive clones | |
|---|---|---|---|
| | Origin of | cDNA library | cDNA library |
| Probe | probe | approach 1¹ | approach 2² |
| 5: E-6 | approach 2 | 0 | 33 |
| Segment 2 | RT-PCR | 0 | 41 |
| 1-1#2; 5-1#1 | approach 1 | 212; 1144 | 6; 43 |
| 2: C-5; 4: D-8 | approach 2 | 0 | 5; 38 |
| 5: E-7 | approach 2 | 0 | 14 |
| Segment 6 | RT-PCR | 0 | 0 |
| 2: B-10 | approach 2 | 0 | 50 |
| Segment 8 | RT-PCR | 6 | 10 |

¹Library from approach 1 had a total of 1364 clone
²Library from approach 2 had a total of 768 clones

Northern Blot Hybridization.

Northern blot analysis was used to correlate each representative sequence with a specific ISAV genomic segment. Total RNA was isolated from CHSE-214 cell monolayers or CHSE-214 cell monolayers infected with ISAV using Trizol (Gibco) as outlined by the manufacturer. The RNA was separated on a 2% agarose gel containing formaldehyde and transferred onto Hybond N⁺ membrane (Amersham) in 10×SSC by capillary action as described in Fourney et al., Focus, 10:5-7 (1992).

The probes used for Northern blot analysis were gel purified, restriction enzyme fragments digested from the plasmids of appropriate cDNA library clones. The probes were labelled with [α³²P]dCTP (NEN) by nick translation (Amersham) and hybridized to the blots at 42° C. for 18 hr in ULTRAhyb™ (Ambion). The membranes were washed 2×5 min in 2×SSC-0.1% SDS at 42° C. and then 2×15 min in 0.1×SSC-0.1% SDS at 42° C. The results were recorded on Kodak X-OMAT AR film.

The probes used in the Northern blot hybridization experiments were derived from four clones constructed using approach 2, one clone constructed using approach 1 and RT-PCR products of the three known segments (see Table 3). A single RNA blot was consecutively probed with each of the eight individual probes. One probe was hybridized to the Northern blot and the results were visualized by autoradiography. The next probe was hybridized to the same Northern blot, the results were visualized and compared with the results from the previous hybridization. By repeating this process with each of the eight probes, each individual probe and its corresponding nucleotide sequence was correlated with a specific RNA segment.

Eight RNA segments were identified; segments 1 and 2 were both approximately 2400 nucleotides in length. The ISAV RNA segment corresponding to each cDNA clone is summarized in Table 5. The genome segments are numbered with respect to their mobility in agarose gels, from the slowest to the fastest and comprise a genome of 14,500 nucleotides.

TABLE 5
RNA segments of ISAV isolate CCBB, their genes and encoded proteins

| | | | | | | Molecular |
|---|---|---|---|---|---|---|
| | | Length of | | | Nascent | weight |
| | | segment¹ | Length of | Encoded | polypeptide | predicted |
| Segment | Clone | (kb) | ORF (bp) | protein | length (aa) | (kDa) |
| 1 | 5:E-6 | 2.4 | 1749 | P1 | — | — |
| 2 | PB1 | 2.4 | 2127 | PB1 | 709 | 80.5 |
| 3 | 1-1#2/5-5#1 | 2.2 | 1851 | NP | 617 | 68.0 |
| 4 | 2:C-5/4:D-8 | 1.9 | 1737 | P2 | 579 | 65.3 |
| 5 | 5:E-7 | 1.6 | 1335 | P3 | 445 | 48.8 |
| 6 | HA | 1.5 | 1185 | HA | 395 | 43.1 |
| 7 | 2:B-10 | 1.3 | 771 | P4 | 257 | 28.6 |
| | | | 441 | P5 | 147 | 16.3 |
| 8 | NS | 1.0 | 705 | P6 | 235 | 26.5 |
| | | | 552 | P7 | 184 | 20.3 |

¹Based on the average length determined from Northern blot hybridization analyses with 2-5 replicates per probe.

Purified cellular RNA was separated on a 2% agarose gel and transferred to a Hybond N+ membrane. Lanes 1-7 & 10 contain cellular RNA from CHSE cells infected with ISA virus isolate CCBB; lane 8 contains cellular RNA from ISA virus isolate ME-01; lane 9 contains cellular RNA from CHSE cells infected with ISA virus isolate NB-99; and lane 11 contains cellular RNA from naive CHSE cells.

The RNA blot was consecutively hybridized with radioactively labeled DNA probes specific for one of the ISA virus RNA segments. The results recorded by autoradiography after the addition of each single probe to the same RNA blot are shown in lanes 1-11. The probes are identified by segment (according to Table 5 above): lane 1, segment 3; lane 2, segment 4; lane 3, segment 6; lane 4, segment 1; lane 5, segment 5; lane 6, segment 7; lane 7, segment 8; lanes 8-11, segment 2. Molecular weight standards on the left are in kbp. The RNA segments are labeled on the right.

Construction of Full-Length Clones of Each ISAV Genome Segment.

Full-length cDNA sequence for each of the ISAV RNA segments, with the exception of segment 1, was generated by rapid amplification of cDNA ends (RACE) PCR using the RLM-RACE kit (Ambion). The PCR products were cloned into either pCR®2.1-TOPO® or pGEM-T as directed by the manufacturers (Invitrogen or Promega, respectively) and then sequenced. AssemblyLIGN 1.0.9b (Oxford Molecular Group) was used to order the overlapping sequenced DNA fragments for construction of the full-length sequence.

PCR primers were designed from the consensus sequence obtained for each ISAV RNA segment and used to amplify full-length cDNA sequence for each segment with the exception of segment 1. The PCR product for each segment was cloned into pGEM-T as directed by the manufacturer (Promega) and DNA from three representative clones was sequenced. The computer programs contained in MacVector™ 6.5.3 (Oxford Molecular Group) were used to identify open reading frames and regions of local similarity. The nucleotide and predicted amino acid sequence for each open reading frame were analyzed by BLAST searches through the National Center for Biotechnology Information server (Altschul et al., 1990; Pearson & Lipman, 1988) or the Influenza database (Los Alamos National Laboratory). The most likely cleavage sites for signal peptidase in HA and 5:E-7 were determined using SignalP V1.1 (Nielsen et al., 1997).

The length of each gene, the corresponding encoded polypeptide(s) and the predicted molecular weights of the translated proteins are summarized in Table 1. Only partial sequence from segment 1 was obtained. The cDNA sequence of segments 1-6 was predicted to encode one open reading frame. Segments 7 and 8 each were predicted to encode two proteins.

Comparison of the cDNA nucleotide and predicted amino acid sequences for the ISA virus genome to those listed in the GenBank and Influenza databases showed that RNA segments 1 and 5 of ISA virus isolate CCBB were unique. RNA segments 2, 3, 4 and 6 were found to encode the putative proteins PB1, NP, PA and HA, respectively. The predicted sequences of the P6 and P7 proteins encoded on RNA segment 8 were similar to the sequences of the two open reading frames (orf) on segment 8 from other ISA virus isolates.

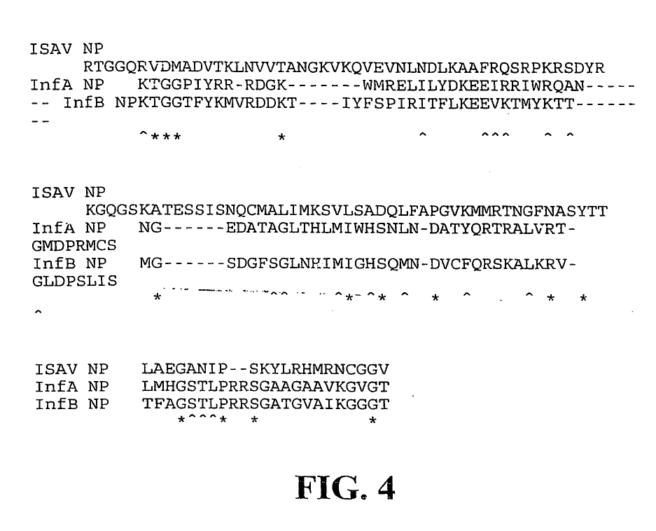

The protein sequence of the partial open reading frame encoded on segment 1 was unique. The predicted amino acid sequence of PB 1, encoded by RNA segment 2, was 82.2 to 84.5% similar to the amino acid sequences of PB1 proteins from Norwegian (AJ002475) and Scottish (AF262392) ISA virus isolates. The assignment of NP to the open reading frame encoded on RNA segment 3 was based on nucleotide sequence similarity to the influenza A NP RNA binding region (see FIG. 4) and to the putative NP sequence described by Snow & Cunningham (2001). The sequence for the CCBB ISA virus NP was highly conserved, sharing 96.6% identity to that reported for the Scottish NP (AJ276858). The predicted protein sequence of P2 from RNA segment 4 had 99% identity to the putative PA sequence (AF306548) described by Ritchie et al. (2001). The nucleotide sequences for segment 5 of the Scottish (AF429988), Norwegian (AF429987) and Maine (AF429986) isolates of ISAV were 76.4, 76.0 and 99.7% similar to the corresponding sequence of ISAV isolate CCBB.

The predicted translation of the open reading frame encoded by RNA segment 6 shared 84.8 to 84.3% identity to the predicted HA protein sequences for ISA virus isolates from Norway (AF302799) and Scotland (AJ276859), and 99.2% identity to the Maine ISA virus isolate (AY059402). The nucleotide sequence for ISAV CCBB segment 7 had 99.6% identity with a reported ISAV sequence (AX083264). The P4 and P5 proteins encoded on segment 7 had 99.2 to 99.3% identity to the translations predicted for orf1 and orf2 from the reported sequence (AX083264). The nucleotide sequence for segment 8 of the Norwegian (AF429990) and ME/01 (AF429989) isolates of ISAV was 88.7-99.9% identical to the corresponding sequence from ISAV isolate CCBB. Our results confirmed that segment 8 encoded two proteins as previously reported by Mjaaland et al. (1997). The amino acid sequence translated from the largest open reading frame was 75.6-97.9% identical to the sequence previously reported for Norwegian (AF262382), Scottish (AJ242016) and Canadian (AJ242016) isolates of ISA virus.

FIG. 4 shows the amino acid sequence alignment of the RNA binding domain of NP from influenza virus A and B with the putative NP RNA binding domain from ISA virus as predicted using the Clustal W system. ISAV NP, aa 189-307 from accession number AF404345; Inf A NP, aa 90-188 from accession number P15675; Inf B NP, aa 149-249 from accession number P04666. Identical amino acids and amino acid residues with similarity in physical and chemical properties are indicated as * and \hat{ }, respectively. The NP RNA binding domain from influenza viruses A and B was taken from Kobayashi et al. (1994).

### Example 5

### Humoral Immune Response

Anti-ISA virus antibodies were generated in Atlantic salmon injected with tissue culture supernatant from ISA virus-infected CHSE cell monolayers. Anti-ISA virus antibodies were also generated in rainbow trout vaccinated with DNA vaccines expressing a ISAV-specific antigens. Mouse polyclonal and monoclonal antibodies (mAbs) to ISA virus were generated by Rob Beecroft (Immuno-Precise Antibodies Ltd.). ISAV-specific immunoreactive antigens were detected by IFAT, ELISA, Western blot and serum neutralization assays.

Indirect Fluorescent Antibody Technique (IFAT)

IFATs were used to screen the ISAV-specific monoclonal antibodies (mAb). CHSE-214 cells infected with ISAV were fixed to a glass slide with 100% acetone, blocked with 3% skim milk buffer, incubated with ISAV-specific mAb 10A3, washed and reacted with TRITC-labelled goat anti-mouse antibody (Sigma). The slide was washed, air dried, and fixed to a glass slide with Cytoseal 60 (Stephens Scientific).

Viral-infected cells were stained a deep red whereas control slides of naive CHSE cells were negative by IFAT with mAb 10A3.

ELISA

The levels of ISAV-specific antibodies in serum from Atlantic salmon infected with ISAV or rainbow trout vaccinated with a DNA vaccine were determined by enzyme-liked immunosorbent assay (ELISA). DNA vaccines tested were pISA-HA (NA), pISA-HA (Nor), pISA-seg7, and pISA-seg8.

ISAV antigen was dried onto wells of an ELISA plate overnight at 37° C. Wells were rinsed three times with PBS/Tween and then serial dilutions of anti-ISAV sera were added in triplicate. After 1 hr, wells were rinsed three times with PBS/Tween. The second antibody, mouse anti-salmon/rainbow trout immunoglobulin (Rob Beecroft), was added to each well. The plates were incubated for 1 hr and then rinsed with PBS/Tween in triplicate. After incubation with the third antibody, goat anti-mouse IgG conjugated to alkaline phosphatase, the wells were washed with PBS/Tween and developer containing p-nitrophenyl phosphate was added. The absorbance was measured at 405 nm.

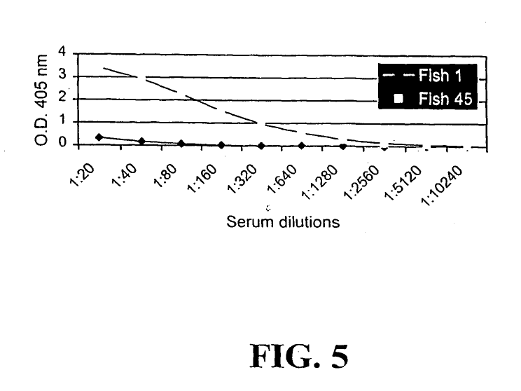

ISAV-specific antibodies were detected in sera from fish injected with either live ISAV (FIGS. 4 and 5) or with DNA vaccines expressing ISAV-specific antigens (FIG. 5).

Antibody studies are conducted in Atlantic salmon vaccinated with an ISAV DNA vaccine, an ISAV recombinant vaccine or a whole killed ISAV vaccine {DNA vaccines: pISA-NP, pISA-Ac, pISA-HA (NA), pISA-HA (Nor), pISA-seg7; recombinant vaccines: rHA-1; whole killed vaccines: 1×, 2× and 4× doses of formalin killed ISAV}. Sera samples are collected from 5 fish/timepoint at 4, 6, 8, 10 and 12 weeks post-vaccination.

FIG. 5 shows the titration of ISAV-specific antibodies from Atlantic salmon infected with ISAV. Fish 1 had not been exposed to ISAV and, thus, the serum was used as a negative control. Fish 45 was injected with ISAV, and the ELISA results indicated that the corresponding serum contained ISAV-specific antibodies. Sera from fish 1 and fish 45 were negative when tested by ELISA using plates coated with CHSE-214 cells.

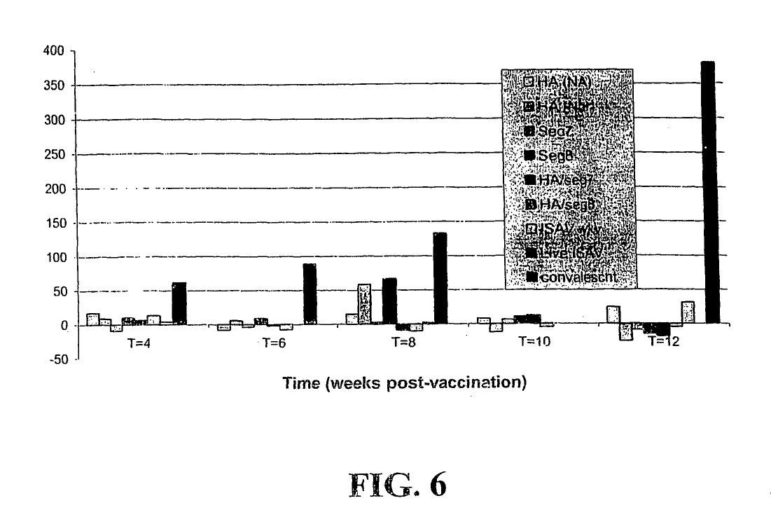

FIG. 6 shows ISAV-specific antibodies in sera obtained from Atlantic salmon infected with ISAV or rainbow trout injected with a nucleic acid encoding an ISAV-specific DNA vaccine. Sera were collected at 4, 6, 8, 10 and 12 weeks post-injection with 1 μg DNA vaccine or post-infection with at least 1×10³ TCID₅₀ live ISAV/fish. Levels of ISAV-specific antibodies were expressed as a percentage of the mAb values to normalize variations between ELISA plates. ISAV-specific antibodies were detected at various times post-treatment. However, the levels of ISAV-specific antibodies were much higher in fish that had been exposed to live virus relative to those injected with the nucleic acid.

SDS-PAGE and Western Blot Analysis:

Whole cell lysates of naive and ISAV-infected CHSE cells as well as purified ISAV were screened for the presence of immunoreactive antigens with mAb 10A3 and sera from Atlantic salmon infected with ISAV. SDS-polyacrylamide gel electrophoresis (PAGE) was carried out by the method of Laemmli (1970). Proteins were solubilized with SDS-polyacrylamide gel electrophoresis (PAGE) sample buffer and separated by SDS-PAGE on 5% stacking gel and 12% resolving gel. Immunoreactive protein bands were visualized by Western blot analysis. Briefly, proteins separated by SDS-PAGE were electrophoretically transferred to nitrocellulose (Bio-Rad Laboratories). The membranes were blocked with 3% skim milk buffer and then incubated with either mAb 10A3 or sera from Atlantic salmon infected with ISAV followed by an incubation with goat anti-mouse immunoglobulin G conjugated to alkaline phosphatase or mouse anti-salmon immunoglobulin (Rob Beecroft), respectively. In the latter case, a final incubation with goat anti-mouse immunoglobulin G conjugated to alkaline phosphatase was required. The immunoreactive proteins were visualized following development with 5-bromo-4-chloro-3-indolyl phosphate and nitroblue tetrazolium.

Immunoreactive polypeptides encoded by the RNA segments were identified by Western blot analysis performed on naive and ISAV-infected CHSE-214 cells (see Table 6 below). Sera, collected from Atlantic salmon injected with live ISA virus, reacted with the 72 and 42 kDa proteins of ISA virus (Table 6). Similar analyses were performed with ISA virus-specific mouse polyclonal and monoclonal antibodies.

TABLE 6
ISAV immunoreactive proteins detected by Western blot analysis

| | Immunoreactive proteins (kDa) | | |
|---|---|---|---|
| Sera | CHSE | CHSE/ISAV | Purified ISAV |
| mAb 10A3 | — | 42 | 42 |
| Mouse polyclonal | — | 42, 36, 25, 15, | 42, 25, 15 |
| | | 11, 9 | |
| Atlantic salmon | — | 72, 42 | Not done |
| convalescent | | | |

*Serum from Atlantic salmon infected with ISAV reacted with a 72 kDa and a 42 kDa protein and was neutralizing. These two proteins are potential vaccine candidates.

Of the six immunoreactive proteins present in the cellular preparation of ISA virus and recognized by the mouse polyclonal sera, three were present in the purified ISA virus sample (42, 25 and 15 kDa; Table 5). Only the 42 kDa protein was recognized by the monoclonal antibody (Table 5). For each serum tested, no reaction was observed with the naive CHSE sample indicating that the immunoreactive proteins were derived from ISA virus.

Serum Neutralization Assay

Ten-fold dilutions of ISAV in PBS were incubated with PBS or serum from naïve or ISAV-infected Atlantic salmon for 1 hr at 15° C. Aliquots of 100 μl of the serum/virus mixture were transferred in quadruplicate to 96-well cell culture plates seeded with CHSE-214 cells, incubated at 15° C. and monitored for CPE. Table 7 summarizes the results.

TABLE 7
Summary of serum neutralization studies

| Sample | Neutralization |
|---|---|
| ISAV/PBS | − |
| ISAV/sera from naïve Atlantic salmon | − |
| ISAV/sera from ISAV-infected Atlantic salmon | + |

*Serum from ISAV-infected Atlantic salmon is neutralizing. Studies to determine if serum from Atlantic salmon infected with ISAV isolate CCBB is neutralizing with the Scottish, Norwegian and Maine isolates of ISAV are underway.

Having illustrated and described the principals of the invention by several embodiments, it should be apparent that those embodiments can be modified in arrangement and detail without departing from the principles of the invention. Thus, the invention includes all such embodiments and variations thereof, and their equivalents.

1. (Amended) a pharmaceutical composition comprising a therapeutically effective amount of an isolated nucleic acid molecule in a pharmaceutically acceptable carrier, wherein the isolated nucleic acid molecule [comprising] comprises at least one of (a) a nucleic acid sequence at least 70% identical to SEQ ID NO: 1; (b) a nucleic acid sequence at least 85% identical to SEQ ID NO: 3; [or] (c) a nucleic acid sequence at least 85% identical to SEQ ID NO: 11; (d) a nucleic acid at least 80% identical to SEQ ID NO: 5, or (e) a nucleic acid at least 80% identical to SEQ ID NO: 9; and wherein the pharmaceutical composition can be used to induce an immune response to ISAV.

2. (Amended) The [nucleic acid molecule] pharmaceutical composition according to claim [1] 34, wherein the nucleic acid sequence is at least 80% identical to SEQ ID NO: [1] 5.

3. (Amended) The [nucleic acid molecule] pharmaceutical composition according to claim [1] 34, wherein the nucleic acid sequence is at least 90% identical to SEQ ID NO: [1] 5, SEQ ID NO: [3] 9, or SEQ ID NO: 11.

4. (Amended) The [nucleic acid molecule] pharmaceutical composition according to claim [1] 34, wherein the nucleic acid sequence is at least 95% identical to SEQ ID NO: [1] 5, SEQ ID NO: [3] 9, or SEQ ID NO: 11.

5. (Amended) The [nucleic acid molecule] pharmaceutical composition according to claim [1] 34, wherein the nucleic acid sequence consists essentially of SEQ ID NO: [1] 5, SEQ ID NO: [3] 9, or SEQ ID NO: 11.

6. (Amended) The [nucleic acid molecule] pharmaceutical composition according to claim [1] 34, wherein the nucleic acid sequence is operably linked to a heterologous nucleic acid comprising an expression control sequence.

7. (Amended) The [nucleic acid molecule] pharmaceutical composition according to claim 6, wherein the nucleic acid molecule encodes an antigenic epitope.

8. (Reiterated) A vector comprising the nucleic acid molecule according to claim 6.

9. (Reiterated) A host cell, comprising the nucleic acid according to claim 6.

10. (Reiterated) The host cell according to claim 9, wherein the cell is a fish cell.

11. (Reiterated) The host cell according to claim 10, wherein the fish cell is from rainbow trout, coho salmon, chinook salmon, amago salmon, chum salmon, sockeye salmon, Atlantic salmon, arctic char, brown trout, cutthroat trout, brook trout, catfish, tilapia, sea bream, seabass, flounder, or sturgeon.

12. (Amended) [A] An isolated nucleic acid comprising at least 100 consecutive nucleotides of SEQ ID NO: [1] 5.

13. (Amended) A transgenic animal, a nucleated cell of which comprises:
an expression control sequence operably linked to a nucleic acid [sequence] molecule comprising (a) a nucleic acid sequence at least 70% identical to SEQ ID NO: 1; (b) a nucleic acid sequence at least 85% identical to SEQ ID NO: 3; [or] (c) a nucleic acid sequence at least 85% identical to SEQ ID NO: 11; (d) a nucleic acid at least 80% identical to SEO ID NO: 5; or (e) a nucleic acid at least 80% identical to SEQ ID NO: 9, and wherein the pharmaceutical composition can be used to induce an immune response to ISAV;
wherein the nucleic acid sequence [at least 70% identical to SEQ ID NO: 1, the nucleic acid sequence at least 85% identical to SEQ ID NO: 3, or the nucleic acid sequence at least 85% identical to SEQ ID NO: 11] encodes an antigenic epitope.

14. (Amended) The transgenic animal according to claim [13] 35, wherein the animal exhibits an increased resistance to infection by infectious salmon anemia virus as compared to a non-transformed animal of the same species.

15. (Amended) The transgenic animal according to claim [13] 35, wherein the animal is an aquaculture animal.

16. (Amended) The transgenic animal according to claim [15] 35, wherein the animal is a fish.

17. The transgenic animal according to claim 16, wherein the fish is rainbow trout, coho salmon, chinook salmon, amago salmon, chum salmon, sockeye salmon, Atlantic salmon, arctic char, brown trout, cutthroat trout, brook trout, catfish, tilapia, sea bream, seabass, flounder, or sturgeon.

18. A method of eliciting an immune response against infections salmon anemia virus in a fish, comprising
introducing into the fish a therapeutically effective amount of the nucleic acid molecule according to claim 6, wherein the nucleic acid molecule encodes an antigenic epitope of infectious salmon anemia virus,
thereby eliciting an immune response against infectious salmon anemia virus in the fish.

19. (Amended) The method according to claim 18, wherein the nucleic acid molecule has a nucleic acid sequence at least 80% identical to SEQ ID NO: [1] 5.

20. (Amended) The method according to claim 19, wherein the nucleic acid molecule has a nucleic acid sequence at least 85% identical to SEQ ID NO: [1] 5.

21. (Amended) The method according to claim 20, wherein the nucleic acid molecule has a nucleic acid sequence at least 90% identical to SEQ ID NO: [1] 5.

22. (Amended) The method according to claim 21, wherein the nucleic acid molecule has a nucleic acid sequence at least 95% identical to SEQ ID NO: [1] 5.

23. (Amended) The method according to claim 22, wherein the nucleic acid molecule has a nucleic acid sequence consisting essentially of SEQ ID NO: [1] 5.

24. (Amended) The method according to claim 18, wherein the nucleic acid sequence is at least 90% identical to SEQ ID NO: [3] 9 or SEQ ID NO: 11.

25. (Amended) The method according to claim 24, wherein the nucleic acid sequence is at least 95% identical to SEQ ID NO: [3] 9 or SEQ ID NO: 11.

26. (Amended) The method according to claim 25, wherein the nucleic acid sequence is at least 95% identical to SEQ ID NO: [3] 9 or SEQ ID NO: 11.

27. (Amended) The method according to claim 26, wherein the nucleic acid sequence consists essentially of SEQ ID NO: [3] 9 or SEQ ID NO: 11.

28. (Reiterated) A method of producing a transgenic fish, comprising contacting a nucleated cell of the fish with an amount of the nucleic acid molecule according to claim 6, wherein the amount of the nucleic acid molecule is sufficient to introduce the nucleic acid molecule into the cell, thereby producing a transgenic fish.

29. (Reiterated) The method according to claim 28, wherein the fish is rainbow trout, coho salmon, chinook salmon, amago salmon, chum salmon, sockeye salmon, Atlantic salmon, arctic char, brown trout, cutthroat trout, brook trout, catfish, tilapia, sea bream, seabass, flounder, or sturgeon.

30. (Reiterated) A polypeptide encoded by the nucleic acid molecule according to claim 1, or a conservative variant thereof.

31. (Reiterated) A method of inducing an immune response in a fish, comprising:
delivering to the fish a therapeutically effective amount of a composition comprising a polypeptide having an amino acid sequence as set forth as SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 15, SEQ ID NO: 17, or SEQ ID NO: 18, an antigenic fragment thereof, or a conservative variant thereof; and
wherein the polypeptide is an antigenic epitope of infectious salmon anemia virus, thereby eliciting an immune response against infectious salmon anemia virus in the fish.

32. (Reiterated) The method according to claim 31 wherein the polypeptide has an amino acid sequence consisting essentially of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 15, SEQ ID NO: 17, or SEQ ID NO: 18.

33. (Reiterated) The method according to claim 31 wherein the polypeptide comprises a fusion protein.

34. (New) The pharmaceutical composition of claim 1, comprising at least one of (a) a nucleic acid sequence at least 85% identical to SEQ ID NO: 11, (b) a nucleic acid at least 80% identical to SEQ ID NO: 5, and (c) a nucleic acid at least 80% identical to SEQ ID NO: 9.

35. (New) A transgenic animal of claim 16, comprising
an expression control sequence operably linked to a nucleic acid molecule comprising at least one of (a) a nucleic acid sequence at least 85% identical to SEQ ID NO: 11, (b) a nucleic acid at least 80% identical to SEQ ID NO: 5, or (c) a nucleic acid at least 80% identical to SEQ ID NO: 9, and wherein the pharmaceutical composition can be used to induce an immune response to ISAV;
wherein the nucleic acid sequence encodes an antigenic epitope.
